(19) United States
(12) Patent Application Publication
(10) Pub. No.: US 2025/0066768 A1
(43) Pub. Date: Feb. 27, 2025

(54) SEQUENCING LIBRARY CONSTRUCTION METHOD AND APPLICATION

(71) Applicants: JIANGSU MICRODIAG BIOMEDICAL TECHNOLOGY CO., LTD., Suzhou Area, Jiangsu (CN); SUZHOU PREMED MEDICAL LABORATORY CO., LTD., Suzhou, Jiangsu (CN)

(72) Inventors: Zhaofen BA, Suzhou (CN); Ruhui SONG, Suzhou (CN); Jiaxin ZHANG, Suzhou (CN); Hao SUN, Suzhou (CN); Tao WANG, Suzhou (CN)

(21) Appl. No.: 18/716,057

(22) PCT Filed: Nov. 18, 2022
(86) PCT No.: PCT/CN2022/132866

§ 371 Date: Jun. 3, 2024

(30) Foreign Application Priority Data
Dec. 1, 2021 (CN) 202111447642.2

(51) Int. Cl. C12N 15/10

(52) U.S. Cl. CPC C12N 15/1068

(57) ABSTRACT

The present disclosure provides an amplification primer for sequencing library construction comprising a primer sequence fragment complementary to a target fragment and a base G ligated to the 5′ end of the primer sequence fragment, wherein the amplification primer and the target fragment are not complementary at the base G. Further disclosed are a method for constructing a sequencing library, a sequencing library, a kit for constructing a sequencing library, a mutant site detection kit, a chromosome ploidy detection kit, a gene fusion detection kit, and a sequencing method.

### CROSS-REFERENCE TO RELATED APPLICATIONS

The present application is an U.S. national phase application under 35 U.S.C. § 371 based upon international patent application No. PCT/CN2022/132866 filed on Nov. 18, 2022, which itself claims priority to Chinese Patent Application No. 2021114476422, entitled “Sequencing library construction method and application”, filed on Dec. 1, 2021, the content of which is incorporated herein by reference in its entirety.

### TECHNICAL FIELD

The present disclosure relates to the field of sequencing technology, and specifically to a sequencing library construction method and application.

### BACKGROUND

High-throughput sequencing is also known as massively parallel sequencing or next-generation sequencing. High-throughput sequencing can sequence multiple target regions of one sample or multiple samples at one time, and its use in clinical applications including pharmacogenomics, genetic disease research and screening, tumor mutation gene detection, and clinical microbiological detection has also gradually received attention.

Whole-genome sequencing has made great progress, but there are still problems related to this technology such as incomplete and inaccurate interpretation databases and high sequencing costs. The high cost and technical complexity of this technology limit the application of whole-genome sequencing. To this terminal, various methods of high-throughput sequencing library construction for target region enrichment for specific genomic regions have been widely developed and applied, thereby improving coverage, and achieving the aim of simplifying the process and reducing costs.

There are two main methods of library construction for targeted enrichment of specific regions: probe hybridization capture and multiplex PCR amplification. Among them, there are currently two main methods of library construction with multiplex PCR products: adding adaptors to the product through a ligation reaction and adding adaptors through two rounds of PCR (the first round with primers having a universal sequence at the 5′ end, and the second round with primers annealing to add adaptors). The library construction method including adding adaptors to multiplex PCR products is a mainstream library construction method.

The ligation effect of a TA sticky end is better than that of a blunt end, and now adding adaptors by TA ligation is mostly used. Some DNA polymerases can catalyze the addition of A bases at the 3′ end of DNA fragments without relying on templates. This discovery laid the foundation for a series of technologies such as TA cloning and next-generation sequencing library construction. However, the current direct catalytic addition of A bases to the 3′ end of DNA fragments is not efficient and is unstable.

### SUMMARY

Based on this, according to various embodiments of the present disclosure, an amplification primer for sequencing library construction is provided, including a primer sequence fragment complementary to a target fragment and a base G ligated to the 5′ end of the primer sequence fragment, wherein the amplification primer and the target fragment are not complementary at the base G.

In one embodiment, the base G is directly ligated to the 5′ end of the primer sequence fragment.

In one embodiment, the base G is a base modified by phosphorylation.

According to various embodiments of the present disclosure, use of the amplification primer described above in preparation of a kit for constructing a sequencing library is provided.

According to various embodiments of the present disclosure, a method for constructing a sequencing library is provided, including:
providing a primer sequence fragment used for performing amplification and library construction on a deoxyribonucleic acid, the primer sequence fragment being complementary to a target fragment,
performing a treatment on the primer sequence fragment to obtain an amplification primer with a base G at the 5′ end, wherein the treatment includes:
ligating a base G to the 5′ end of the primer sequence fragment if the base at the 5′ end of the primer sequence fragment is not G, or
ligating or not ligating a base G to the 5′ end of the primer sequence fragment if the base at the 5′ end of the primer sequence fragment is G;
performing cyclic amplification on the deoxyribonucleic acid using the amplification primer with a base G at the 5′ end, to obtain an amplification fragment with a base C at the 3′ end;
adding a base A to the 3′ end of the amplification fragment; and
ligating an adaptor containing a T sticky end to the amplification fragment with base A added to the 3′ end, wherein the adaptor contains a sequence required by a sequencing platform.

In one embodiment, the method further includes: phosphorylating the base G at the 5′ end of the amplification primer before performing amplification on the deoxyribonucleic acid.

In one embodiment, the deoxyribonucleic acid is selected from the group consisting of a sample DNA, a sample plasmid, and a deoxyribonucleic acid obtained by reverse transcription of a sample RNA.

According to various embodiments of the present disclosure, a sequencing library constructed by the above method for constructing a sequencing library is provided.

According to various embodiments of the present disclosure, a kit for constructing a sequencing library is provided, including the amplification primer described above.

In one embodiment, the kit further includes at least one of a reagent for adding base A, a reagent for adding adaptors, a reagent for PCR amplification and a reagent for purification.

According to various embodiments of the present disclosure, a sequencing method is provided, including: constructing a sequencing library for a sample using the above method for constructing a sequencing library, and performing sequencing.

In one embodiment, the sequencing method is used to perform on the sample anyone of mutation site detection, chromosome ploidy detection, gene fusion detection, and pathogenic microorganism detection.

In one embodiment, the mutation site includes at least one of intestinal cancer mutation sites, cervical cancer mutation sites, and urothelial cancer mutation sites.

In one embodiment, the chromosome ploidy includes at least one of the chromosome ploidy of intestinal cancer, the chromosome ploidy of cervical cancer, and the chromosome ploidy of urothelial cancer.

In one embodiment, the gene fusion includes at least one of EML4-ALK gene fusion, CD74-ROSI gene fusion, CCDC6-RET gene fusion, NCOA4-RET gene fusion, and TPM3-NTRK1 gene fusion.

In one embodiment, the pathogenic microorganism includes at least one of influenza A virus, influenza B virus and SARS-CoV-2.

In one embodiment, the sequencing is high-throughput sequencing.

According to various embodiments of the present disclosure, a mutation site detection kit is provided, including the amplification primer described above.

According to various embodiments of the present disclosure, a chromosome ploidy detection kit is provided, including the amplification primer described above.

According to various embodiments of the present disclosure, a gene fusion detection kit is provided, including the amplification primer described above.

According to various embodiments of the present disclosure, a pathogenic microorganism detection kit is provided, including the amplification primer described above.

According to various embodiments of the present disclosure, a TA cloning method is provided, including the steps of: performing PCR amplification using the amplification primer described above to add a base A to the end of an amplification product.

According to various embodiments of the present disclosure, a method for diagnosing a disease in a subject is provided, including collecting a biological sample from the subject, and performing sequencing on the biological sample using the sequencing method described above, wherein the disease is selected from a cancer or a microorganism infection.

The details of one or more embodiments of the present disclosure are set forth in the accompanying drawings and the description below. Other features, objects and advantages of the present disclosure will become apparent from the description, the accompanying drawings, and the claims.

### BRIEF DESCRIPTION OF THE DRAWINGS

To better describe and illustrate the embodiments and/or examples of the disclosure disclosed herein, reference may be made to one or more accompanying drawings. The additional details or examples used to describe the accompanying drawings are not to be construed as limiting the scope of any one of the disclosed disclosures, the presently described embodiments and/or examples, and the presently understood preferred mode of the disclosure.

FIG. 1 is a schematic diagram of the amplicon library construction process for DNA using conventional primers and primers of the present disclosure using high-fidelity multiple enzymes, according to an example of the present disclosure.

FIG. 2 is a schematic diagram of the amplicon library construction process for DNA using conventional primers and primers of the present disclosure using non-high-fidelity multiple enzymes, according to an example of the present disclosure.

FIG. 3 is a schematic diagram of the amplicon library construction process for RNA using conventional primers and primers of the present disclosure using high-fidelity multiple enzymes, according to an example of the present disclosure.

FIG. 4 is a schematic diagram of the amplicon library construction process for RNA using conventional primers and primers of the present disclosure using non-high-fidelity multiple enzymes, according to an example of the present disclosure.

### DETAILED DESCRIPTION OF THE EMBODIMENTS

In order to facilitate the understanding of the present disclosure, the present disclosure will be described more fully hereinafter with reference to the related accompanying drawings. Various embodiments of the present disclosure are presented in the accompanying drawings. However, the present disclosure may be embodied in many different forms and is not limited to the embodiments described herein. Rather, these embodiments are provided so that the understanding of the content of the present disclosure will be more thorough.

All technical and scientific terms used herein have the same meaning as commonly understood by one of ordinary skill in the art to which the present disclosure applies, unless otherwise defined. The terms used in the specification of the present disclosure herein are for the purpose of describing specific embodiments only and are not intended to limit the present disclosure.

The addition efficiency of 3′ end dA varies depending on the template. For example, the addition efficiency of 3′ end dA depends on the nucleotide base composition of the 3′ end of DNA, and fluctuates between 4% and 75%. The corresponding A—addition efficiency in the case where the 3′ end nucleotide of DNA is C, T, G, or A bases is 80-85%, 75-80%, 45-50%, or 20-30%, respectively.

Therefore, the efficiency of adding A to DNA fragments with different ends is different.

It is resolved in the present disclosure that TA ligation efficiency is improved by adding G to the 5′ end of the forward and reverse primers (it would not be needed to be added if the primer originally has a G at the 5′ end) so that the 3′ ends of the upper and lower complementary strands of the amplicon are both C, thus guaranteeing the addition of A to each chain with a equal probability and a maximized efficiency of 80-85% due to the 3′ end being C, in a process of catalyzing the addition of A at the 3′ end in a non-template-dependent manner.

Through the present disclosure, a higher A-addition efficiency is ensured, and more templates can be added with adaptors through TA ligation, which improves the library conversion rate; and also reduces the processes such as terminal repairing, and reduces the decline of DNA recovery rate caused by complicated operations. This method is more conducive to the detection of low-frequency mutations.

Also, the cloning efficiency of this method is also better than that of conventional methods in the application of TA cloning of PCR products.

In first aspect, embodiments of the present disclosure provide an amplification primer for sequencing library construction, including a primer sequence fragment complementary to a target fragment and a base G ligated to the 5′ end of the primer sequence fragment, wherein the amplification primer and the target fragment are not complementary at the base G.

As used herein, the term “complementary” means that two nucleic acid sequences are capable of forming hydrogen bonds between each other according to the base pairing principle (Waston-Crick principle) and thereby forming a duplex. In the present disclosure, the term “complementary” includes “substantially complementary” and “completely complementarity”. As used herein, the term “completely complementarity” means that all bases in one nucleic acid sequence are capable of pairing with bases in the other nucleic acid strand without the presence of mismatches or gaps. As used herein, the term “substantially complementarity” means that a majority of the bases in one nucleic acid sequence is capable of pairing with bases in the other nucleic acid strand with mismatches or gaps (e.g., a mismatch or gap of one or more nucleotides) allowed to be present. Generally, under conditions that allow hybridization, annealing, or amplification of the nucleic acids, two nucleic acid sequences that are “complementary” (e.g., substantially complementary or completely complementary) will selectively/specifically hybridize or anneal to form a duplex.

As used herein, the terms “target fragment”, “target amplification fragment” and “target sequence” refer to the target nucleic acid sequence to be amplified. In the present disclosure, the terms “target fragment”, “target amplification fragment” and “target sequence” have the same meaning and can be used interchangeably. It is easy to understand that the target fragment is specific for the sample sequence. In other words, under conditions that allow nucleic acid hybridization, annealing or amplification, the amplification primer only hybridizes or anneals to a specific target fragment to amplify the specific fragment, but does not hybridize or anneal to other nucleic acid sequences.

When the amplification primer is complementary to the target fragment and the primer sequence fragment is complementary to the target fragment, the amplification primer and the target fragment are not complementary at base G.

In some embodiments, base G is directly ligated to the 5′ end of the complementary sequence without other bases spaced in between.

In some embodiments, the base G is abase modified by phosphorylation. The 5′ end of the primer is modified by phosphorylation to ensure the phosphorylation state of the 5′ ends of the double-strand product. Phosphorylation of the 5′ end of the primer can reduce the phosphorylation reaction and purification steps in library construction, thus saving time and reducing molecular loss caused by multiple operating steps. Adding G to the 5′ end of the primer in combination with phosphorylation of the 5′ end can improve the library conversion rate and reduce the loss rate.

In a second aspect, the embodiments of the present disclosure provide the use of the above amplification primer in preparation of a kit for constructing a sequencing library, so as to ensure that the kit achieves a higher A-adding efficiency, so that more templates can be added with adaptors through TA ligation, which improves the library conversion rate; and the kit also reduces processes such as terminal repairing, and reduces the decline of DNA recovery rate caused by complicated operations.

In a third aspect, the embodiments of the present disclosure provide a method for constructing a sequencing library, including:
providing a primer sequence fragment used for performing amplification and library construction on a deoxyribonucleic acid, the primer sequence fragment being complementary to a target fragment, and
performing a treatment on the primer sequence fragment to obtain an amplification primer with a base G at the 5′ end, wherein the treatment includes:
ligating a base G to the 5′ end of the primer sequence fragment if the base at the 5′ end of the primer sequence fragment is not G, or
ligating or not ligating a base G to the 5′ end of the primer sequence fragment if the base at the 5′ end of the primer sequence fragment is G;
performing cyclic amplification on the deoxyribonucleic acid using the amplification primer, to obtain an amplification fragment with a base C at the 3′ end;
adding a base A to the 3′ end of the deoxyribonucleic acid; and ligating an adaptor containing a T sticky end to the amplification fragment with base A added to the 3′ end, wherein the adaptor contains a sequence required by a sequencing platform.

On the basis that the addition efficiency of 3′ end dA varies depending on the template, when the nucleotide base at the 3′ end is a C base, the corresponding A-addition efficiency is the highest. In the above sequencing library construction method of the present disclosure, base G is added to the 5′ end (it would not be needed to be added additionally if G is originally present) of the PCR primer to ensure that the 3′ end of the PCR product is C, ensuring that each product molecule has the same, higher A-addition efficiency. Since the efficiency of adding A to the ends of the product is improved, the ligation efficiency is improved in the reaction of adding adaptor in TA ligation. Compared with conventional library construction methods, the method of introducing modifications at the primer ends to obtain the amplification product which was conducive to the addition of A reduces the terminal repairing and other processes after amplification, and reduces decline of DNA recovery rate caused by complicated operations, has simple steps, saves reagents, and has higher ligation efficiency to adaptor. This method can also be used for PCR products in TA cloning, mutation site detection, chromosome ploidy detection, gene fusion detection, pathogenic microorganism detection, and other detection methods using sequencing.

The above deoxyribonucleic acid is selected from the group consisting of a sample DNA, a sample plasmid, and a deoxyribonucleic acid obtained by reverse transcription of a sample RNA.

In some embodiments, the method for constructing a sequencing library further includes the step of amplifying the ligation product using an amplification primer of the adaptor to achieve pre-amplification of the library.

In a fourth aspect, an embodiment of the present disclosure provide a sequencing library constructed by the above method for constructing a sequencing library.

In a fifth aspect, an embodiment of the present disclosure provide a kit for constructing a sequencing library.

A kit for constructing a sequencing library includes the amplification primers of any of the above embodiments.

The term “kit” refers to any article (e.g., package or container) that includes at least one device. The kit may further include any one of instructions for use, supplementary reagents, components, or assemblies, which are for use in the methods described herein or steps thereof.

Optionally, the kit also includes any one or more of a reagent for adding A, a reagent for adding adaptors, a reagent for PCR amplification and a reagent for purification.

Reagents for adding A include enzymes and dATP.

In some embodiments, the enzyme used to add A to 3′ end is any one or more selected from the group consisting of klenowex-enzyme, Taq enzyme, and klenowex-enzyme with Taq enzyme. Alternatively, the addition of A to 3′ end and ligation of the adaptor may be performed in one reaction system, or the addition of A to 3′ end is performed followed by purification before ligation to the adaptor. Performing in one reaction system means that the sequencing adaptor is directly ligated without purification after adding A to the 3′ end. Taq enzyme is preferred to be used for the addition of A to 3′ end.

Reagents for adding adaptors include ligases and adaptors.

Ligase is any one or more selected from the group consisting of HiFi Taq DNA ligase, T4 RNA ligase 2, SplintR® Ligase, 9° N™ DNA ligase, Taq DNA ligase, T7 DNA ligase, T3 DNA ligase, Electro Ligase, Blunt end/TA ligase premix, instant sticky ligase premix, T4 DNA ligase, Circligase ssDNA ligase, 5′AppDNA/RNA thermostable ligase.

The adaptor has a T base protruding at the 3′ end, which is used for complementary pairing with the sample DNA fragment after adding A.

The reagents used for PCR amplification may be any one or more selected from the group consisting of DNA polymerases, dNTPs and DNA polymerase buffers.

Furthermore, the DNA polymerase is a combination of one or more enzymes selected from the group consisting of vent DNA polymerase, T7 DNA polymerase, Bsu DNA polymerase, T4 DNA polymerase, Klenow fragment, DNA polymerase I (E. coli), Therminator™ DNA polymerase, SulfolobusDNA polymerase IV, phi29 DNA Polymerase, Bst 2.0 DNA Polymerase, BstDNA Polymerase, Deep VentR®DNA Polymerase, Deep VentRThIDNA Polymerase, VentR®DNA Polymerase, EpiMark® Hot Start Taq DNA Polymerase, LongAmp® Hot Start Taq DNA Polymerase, LongAmp® Taq DNA Polymerase, Taq DNAPolymerase Large Fragment, OneTaq® Hot Start DNA Polymerase, Phusion® Hot Start Flex DNA Polymerase, Phusion® Ultra-Fidelity DNA Polymerase, and Q5@Hot Start Q5@ultra-fidelity DNA polymerase, etc.

Further, dNTPs may have a concentration of 2.5 mM, 10 mM, etc. DNA polymerase buffer is the best matching buffer selected for the selected polymerase.

The step of purifying DNA fragments may be performed by conventional methods in the art, for example, by using purification magnetic beads. Alternatively, reagents used for purification may include: 1.8× magnetic beads, 0.9× magnetic beads, and EB solution.

In the kit of the present disclosure, the reagents are preferably packaged individually, but they can also be mixed-packaged on the premise of not affecting the implementation of the present disclosure.

In a sixth aspect, a sequencing method is provided, including: constructing a sequencing library for a sample using the method for constructing a sequencing library of the above embodiments, and performing sequencing.

In the method of the present disclosure, the sample may be any sample to be sequenced. For example, in certain embodiments, the sample contains or is DNA (e.g., genomic DNA or cDNA). In certain embodiments, the sample contains or is RNA (e.g., mRNA). In certain embodiments, the sample contains or is a mixture of nucleic acids (e.g., a mixture of DNA, a mixture of RNA, or a mixture of DNA and RNA).

In the methods of the present disclosure, the sequence to be sequenced is not limited by its sequence composition or length. For example, the sequence to be sequenced may be DNA (e.g., genomic DNA or cDNA) or RNA molecules (e.g., mRNA). Furthermore, the target nucleic acid sequence to be detected may be single-stranded or double-stranded.

When the sample to be detected or the sequence to be sequenced is RNA, it is preferred to perform a reverse transcription reaction to obtain cDNA which is complementary to the mRNA before performing the method of the present disclosure. For a detailed description of the reverse transcription reaction, see, for example, Joseph Sam-brook, et al., Molecular Cloning, A Laboratory Manual, Cold Spring Harbor Laboratory Press, Cold Spring Harbor, N. Y. (2001).

The sample to be sequenced or the sequence to be sequenced may be obtained from any sources, including but not limited to prokaryotes (such as bacteria), eukaryotes (such as protozoa, parasites, fungi, yeast, plants, animals including mammals and humans) or viruses (such as Herpes virus, HIM influenza virus, Epstein-Barr virus, hepatitis virus, poliovirus, etc.) or viroids. The sample to be sequenced or the sequence to be sequenced may also be a nucleic acid sequence in any form, such as a genome sequence, an artificially isolated or fragmented sequence, a synthetic sequence, etc.

In some embodiments, the sample to be sequenced is blood, serum, plasma, cell culture supernatant, saliva, semen, tissues or tissue lysate.

In some embodiments of the present disclosure, the sequencing method is high-throughput sequencing, also known as next-generation sequencing (“NGS”). Next-generation sequencing generates thousands to millions of sequences simultaneously in a parallel sequencing process. NGS is distinguished from “Sanger sequencing” (first-generation sequencing) in that the latter is based on the electrophoretic separation of chain termination products in a single sequencing reaction. Sequencing platforms for NGS that may be used in the present disclosure may be commercially available, including but not limited to Illumina MiniSeq, NextSeq 550, life platform, etc.

This method may be used for non-diagnostic purposes, such as used for genetic research, and researches in racial distribution, human evolution and other fields (typically such as SNP applications).

In some embodiments, the sequencing methods of the above embodiments of the present disclosure may be used for samples for mutation site detection, chromosome ploidy detection, gene fusion detection, pathogenic microorganism detection and other purposes.

In some embodiments, the mutation site includes at least one of an intestinal cancer mutation site, a cervical cancer mutation site, and an urothelial cancer mutation site.

In some embodiments, the chromosome ploidy includes at least one of the chromosome ploidy of intestinal cancer, the chromosome ploidy of cervical cancer, and the chromosome ploidy of urothelial cancer.

In some embodiments, the gene fusion includes at least one of EMLA-ALK gene fusion, CD74-ROSI gene fusion, CCDC6-RET gene fusion, NCOA4-RET gene fusion, and TPM3-NTRK1 gene fusion.

In some embodiments, the pathogenic microorganism includes at least one of influenza A virus, influenza B virus and SARS-CoV-2.

In a seventh aspect, an embodiment of the present disclosure provide a mutation site detection kit, including the amplification primer of any of the above embodiments.

In an eighth aspect, an embodiment of the present disclosure provide a chromosome ploidy detection kit, including the amplification primer of any of the above embodiments.

In a ninth aspect, embodiments of the present disclosure provide a gene fusion detection kit, including the amplification primer of any of the above embodiments.

In a tenth aspect, embodiments of the present disclosure provide a pathogenic microorganism detection kit, including the amplification primer of any of the above embodiments.

In an eleventh aspect, a TA cloning method is provided, including the steps of: performing PCR amplification using the amplification primer of any of the above embodiments, thereby adding A to the terminal of an amplification product and improving the efficiency of adding A to the end of the PCR product.

In a twelfth aspect, a method for diagnosing a disease in a subject is provided, including collecting a biological sample from the subject, and performing sequencing on the biological sample using the sequencing method any of the above embodiments, wherein the disease is selected from a cancer or a microorganism infection.

### Example 1: Experimental Procedures and Methods

### (1) NGS Related Experiment

1. Library Construction with Multiplex PCR Products Using Traditional and Application Methods.

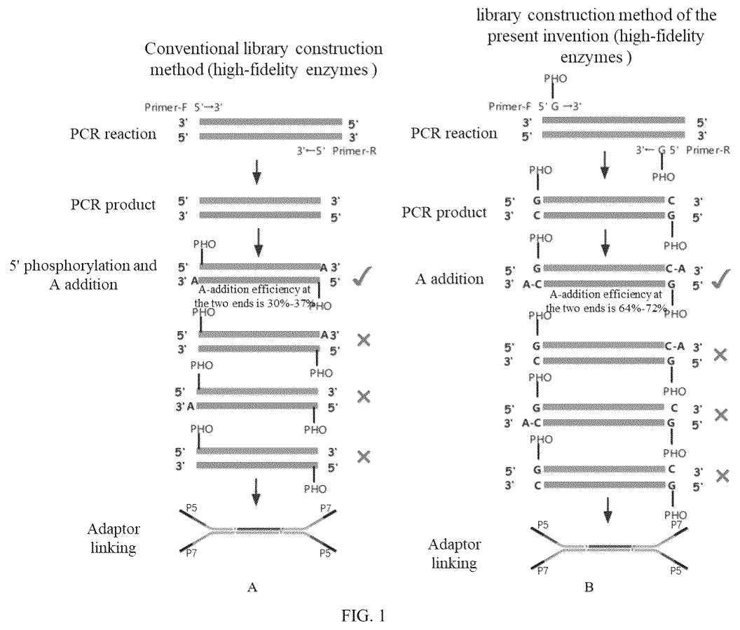

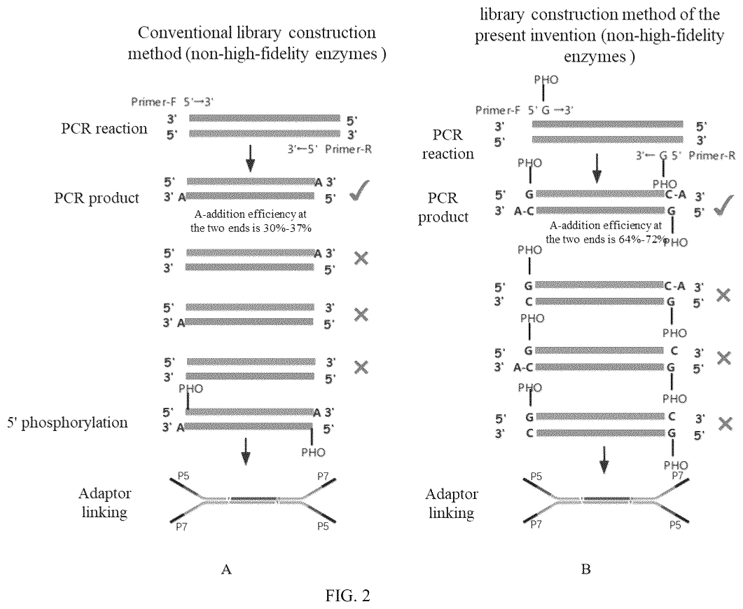

The library construction process for DNA is shown in FIGS. 1 and 2.

FIG. 1: A. When constructing a library with a high-fidelity enzyme using a conventional method, the PCR product is subjected to 5′ end phosphorylation and A-addition after PCR reaction, with A-addition efficiency at the two ends of only 30%-37%, and then an adaptor is ligated. B. When constructing a library with a high-fidelity enzyme using the method of the present disclosure, A is directly added to the PCR product after PCR reaction, with A-addition efficiency at the two ends of 64%-72%, and then an adaptor is ligated.

FIG. 2: A. When constructing a library with a non-high-fidelity enzyme using a conventional method, the PCR product is subjected to A-addition after the PCR reaction, with A-addition efficiency at the two ends of only 30%-37%, and then the 5′ end is phosphorylated and the adaptor is ligated. B. When constructing a library with a non-high-fidelity enzyme using the method of the present disclosure, after the PCR reaction, A has already been added to the 3′ end of the PCR product and phosphorylation has been done at the 5′ end, with A-addition efficiency at the two ends of 64%-72%, and the adaptor can be ligated directly.

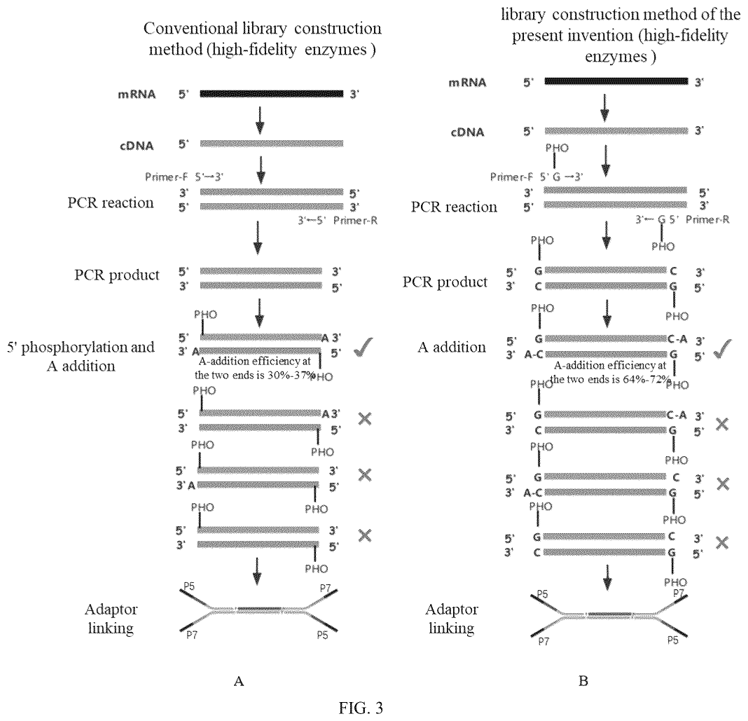

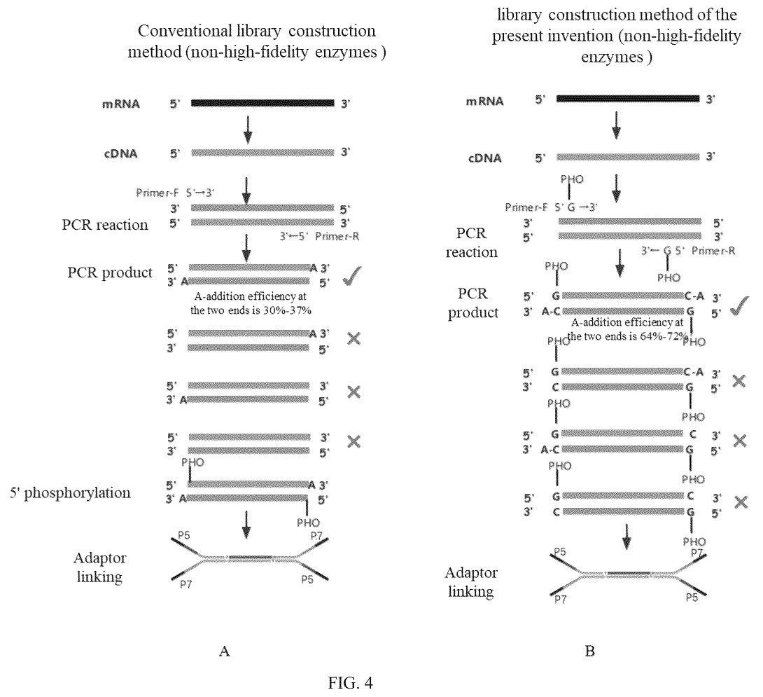

The RNA library construction process for RNA is shown in FIGS. 3 and 4.

FIG. 3: A. When constructing a library with a high-fidelity enzyme using a conventional method, the mRNA is reverse transcribed into cDNA before performing PCR reaction, then the PCR product is subjected to 5′ end phosphorylation and A-addition, with A-addition efficiency at the two ends of only 30%-37%, and then the adaptor is ligated; B. When constructing a library with a high-fidelity enzyme using the method of the present disclosure, the mRNA is reverse transcribed into cDNA before performing PCR reaction, then A is directly added to the PCR product, with A-addition efficiency at the two ends of 64%-72%, and then the adaptor is ligated.

FIG. 4: A. When constructing a library with a non-high-fidelity enzyme using a conventional method, the mRNA is reverse transcribed into cDNA before performing PCR reaction, and the PCR product is subjected to A-addition after PCR reaction, with A-addition efficiency at the two ends of only 30%-37%, and then the 5′ end is phosphorylated and the adaptor is ligated.; B. When constructing a library with a high-fidelity enzyme using the method of the present disclosure, the mRNA is reverse transcribed into cDNA before performing PCR reaction, and after the PCR reaction, A has already been added to the 3′ end of the PCR product and phosphorylation has been done at the 5′ end, with A-addition efficiency at the two ends of 64%-72%, and the adaptor can be ligated directly.

### 2. Detailed Experiments

### 1) Sample Collection:

Plasma samples: Whole blood was collected in EDTA blood collection tubes, and centrifuged at room temperature within 1 hour to obtain plasma. Centrifugation conditions: centrifuged at 1,500 g for 10 minutes. The supernatant was aspirated and collected, and then centrifuged again at 15,000 g for 10 minutes. The supernatant was aspirated and collected, which was the separated plasma.

Cervical exfoliated cell samples: Cervical exfoliated cell samples were collected in a ThinPrep cervical exfoliated cell preservation solution, stored at 4° C., and centrifuged at 12,000 rpm for 10 minutes. The supernatant was discarded, obtaining the cervical exfoliated cell pellet.

Urine samples: About 15-25 mL of morning urine was collected in a clean and dry disposable urine cup, and centrifuged at 500 g for 10 minutes. The supernatant was discarded, obtaining the urothelial cell pellet.

Bowel cancer tissue samples: Fresh bowel cancer tissue, preserved in RNAlater.

### 2) Extraction and Concentration Determination of Sample DNA:

Plasma DNA extraction: Plasma samples were subjected to extraction using a QIAAMP Circulating nucleic acid kit (QIAGEN-55114).

Extraction of gDNA from cervical exfoliated cells: CWBio Blood Genomic DNA Mini Kit (CW2087M) was used, and the starting volume of each sample was 2 mL.

DNA extraction from urine samples: CWBio Blood Genomic DNA Mini Kit (CW2087M) was used to extract DNA from urine samples.

Extraction of bowel cancer FFPE samples: PureLink™ Genomic DNA Mini Kit (Invitrogen) was used to extract DNA from bowel cancer tissue.

The concentration of DNA was determined by Qubit.

### 3) PCR Targeted Enrichment:

High-fidelity and non-high-fidelity multiplex PCR enzymes were used for PCR. The reaction system and conditions were in accordance with the instructions of the multiplex enzymes.

### 4) Subsequent Reactions:

For details of reactions such as A-addition, phosphorylation, and ligation, see the Examples.

### 5) Quality Evaluation and Concentration Determination of the Library:

The library concentration was determined by Qubit, and the reagent used was Qubit dsDNA HS Assay Kit, 500 assay (invitrogen/Q32854).

Library quality control: KAPA Library Quantification Kit Illumina® Platforms (KK4824) was used to evaluate effective rate of the library.

### 6) Quality Inspection of the Library:

The resulting library was subjected to 2100 quality inspection using a high-sensitivity DNA kit 10 (Agilent/5067-4626).

### 7) Sequencing On-Machine for Mixed Samples:

Sequencing was performed using the next 500 or novaseq of the Illumina platform.

### 8) Off-Machine Sequencing Library Analysis:

The test data were analyzed for mutation and ploidy using Jiangsu MicroDiag detection software and chromosome abnormality analysis software respectively, and the pathogenic chromosomes were analyzed using MicroDiag's in-house method.

### (2) TA Cloning Related Experiments

Purg gene of zebrafish was PCR amplified, and the product was TA cloned. The TA cloning efficiency of PCR products with conventional primers and different modified primers were examined.

Zebrafish were purchased from Suzhou Murui Biotech Co., Ltd. For DNA extraction, firstly, the tail fins cut into pieces were homogenized using a tissue homogenizer, and then DNA was extracted using a DNeasy Blood & Tissue Kit (QIAGEN, Cat. No.: 69582), the concentration of DNA was measured by Qubit.

The kit used in the PCR reaction was Takara LA Taq DNA Polymerase (Takara Biotech), the amount of DNA template in the PCR reaction was 500 ng, and amplification was carried out according to the PCR reaction system and conditions recommended in the instructions. QIAquick PCR Purification Kit (QIAGEN) was used for purification and recovery of PCR products. 2100 was used to check whether the size of the PCR fragment was correct and whether there were dimers. If there were dimers and non-specific amplification, gel cutting recovery should be performed with a GenElute™ gel recovery kit (Sigma-Aldrich). The purified product concentration was determined using Qubit.

The kit used in TA cloning was Mighty TA-cloning Kit (Takara Biotech). AT vector and the PCR product to be inserted were mixed at a molar ratio of 1:3 to ensure that the volume was 5 μL total. Then 5 μL of Ligation Mighty Mix was added and mixed well gently. The reaction was performed at 16° C. for 30 minutes, and all added to 100 μl of competent cells (E. coli, DH5a) for transformation. Then the cells were spread on an L-agar plate containing X-Gal, IPTG, and Amp, and cultured at 37° C. overnight. The Colony was directly subjected to PCR to confirm the recombinants.

### Example 2: Library Effective Rate

MRC-5 cell DNA was amplified using the four kinds of primers in Table 1 to verify the library effective rate of amplicon library construction using different primers.

The primer sequences and modifications are shown in Table 17, including 4 kinds of primers, ordinary primers (1), primer (2) that is the ordinary primers with phosphorylated 5′ end, primer (3) that is is the ordinary primers with G added to 5′ end, and primers (4) that is ordinary primers with G added to 5′ end and phosphorylated, the concentration of each primer in each kind of primer pool was 400 nM. Multiplex PCRQIAGEN multiple enzyme was used to perform the PCR reaction in a system as follows. 10 μL of primer mixture was added, 30 ng of template was added, the other components were added following the instructions, and the system was finally made up to 50 μL with water. The reaction conditions were set according to the instructions. The product was purified using a PCR product purification kit (QIAGEN), and eluted in 20 μL. For reactions such as base A-addition, adaptor ligation, and library pre-amplification, see patent CN103298955B.

Qubit dsDNA HS Assay Kit, 500 assay (invitrogen/Q32854) and KAPA Library Quantification Kit Illumina® Platforms (KK4824) were used to evaluate the effective rate of libraries constructed with different primers. The actual effective concentration was the library concentration measured using the KAPA Library Quantification Kit. Library effective rate=actual effective concentration/library concentration (Qubit) See Table 1 for details

TABLE 1
Library effective rate for library construction using four kinds of primers

| | | | | Primers with phosphorylated | | | Primers with G added to | | | Primers with G added to 5′ | | |
|---|---|---|---|---|---|---|---|---|---|---|---|---|
| | Ordinary primers | | | 5′ end | | | 5′ end | | | end and phosphorylated | | |
| | | Actual | Library | | Actual | Library | | Actual | Library | | Actual | Library |
| | Library | effec- | effec- | Library | effec- | effec- | Library | effec- | effec- | Library | effec- | effec- |
| Sample | conc. | tive | tive | conc. | tive | tive | conc. | tive | tive | conc. | tive | tive |
| type | (Qubit) | conc. | rate | (Qubit) | conc. | rate | (Qubit) | conc. | rate | (Qubit) | conc. | rate |
| HD734 | 2.16 | 1.38 | 63.89% | 2.12 | 1.79 | 84.43% | 2.23 | 1.99 | 89.24% | 2.65 | 2.53 | 95.47% |
| HD752 | 2.11 | 1.25 | 59.24% | 2.35 | 2.05 | 87.23% | 2.36 | 2.13 | 90.25% | 2.78 | 2.62 | 94.24% |
| HD813 | 2.26 | 1.61 | 71.24% | 2.26 | 2.01 | 88.94% | 2.51 | 2.33 | 92.83% | 2.91 | 2.78 | 95.53% |
| HD815 | 2.84 | 1.72 | 60.56% | 2.51 | 2.16 | 86.06% | 2.31 | 2.09 | 90.48% | 2.84 | 2.69 | 94.72% |
| Ref. A | 2.31 | 1.11 | 48.05% | 2.11 | 1.85 | 87.68% | 2.21 | 2.01 | 90.95% | 2.52 | 2.43 | 96.43% |
| Ref. B | 2.16 | 1.36 | 62.96% | 2.23 | 1.95 | 87.44% | 2.33 | 2.12 | 90.99% | 2.51 | 2.41 | 96.02% |
| Ref. C | 2.22 | 1.18 | 53.15% | 2.41 | 2.11 | 87.55% | 2.54 | 2.29 | 90.16% | 2.55 | 2.44 | 95.69% |
| Intestinal | 1.97 | 1.12 | 56.85% | 1.91 | 1.61 | 84.29% | 2.31 | 2.04 | 88.31% | 2.63 | 2.49 | 94.68% |
| cancer sample | | | | | | | | | | | | |
| Healthy person | 1.88 | 1.34 | 71.28% | 1.99 | 1.66 | 83.42% | 2.26 | 2.01 | 88.94% | 2.61 | 2.51 | 96.17% |
| plasma sample | | | | | | | | | | | | |
| Cervical exfoliated | 3.02 | 1.98 | 65.56% | 3.12 | 2.55 | 81.73% | 3.2 | 2.79 | 87.19% | 3.33 | 3.10 | 93.09% |
| cell samples | | | | | | | | | | | | |
| urothelial | 2.88 | 2.11 | 73.26% | 3.01 | 2.39 | 79.40% | 3.12 | 2.64 | 84.62% | 3.29 | 2.99 | 90.88% |
| cancer sample | | | | | | | | | | | | |
| Healthy person's | 2.98 | 2.03 | 68.12% | 2.56 | 2.03 | 79.30% | 2.94 | 2.68 | 91.16% | 3.41 | 3.27 | 95.89% |
| urine sample | | | | | | | | | | | | |

Result analysis: the library concentration and effective rate of the libraries constructed with the four kinds of primers were normal; the library effective rate for primers with G added to 5′ end and phosphorylated was better than that of the primers with G added to 5′ end; the library effective rate of the primers with G added to 5′ end was better than that of the primers with phosphorylated 5′ end; the library effective rate of the primers with phosphorylated 5′ end is better than that of ordinary primers.

### Example 3: Detection of Sites of Commercial gDNA Reference

Positive reference HD734 from Horizon and negative reference HD752 (100% Wildtype) were used to verify the detection effects of different primers for library construction.

The primer sequences and modifications are shown in Table 17, including 4 kinds of primers, ordinary primers (1), primer (2) that is the ordinary primers with phosphorylated 5′ end, primer (3) that is is the ordinary primers with G added to 5′ end, and primers (4) that is ordinary primers with G added to 5′ end and phosphorylated, the concentration of each primer in each kind of primer pool was 400 nM. A kit (Phusion U multiplex PCR master mix, Thermo Scientific) was used to perform the multiplex PCR reaction in a PCR reaction system as follows. 10 μL of primer mixture was added, 30 ng of template was added, the other components were added following the instructions, and the system was finally made up to 50 μL with water. The reaction conditions were set according to the instructions. The product was purified using a PCR product purification kit (QIAGEN), and eluted in 20 μL The product obtained using the first primers needed to be phosphorylated. T4 PNK enzyme from NEB was used, and the reaction system and conditions were set according to the instructions of the enzyme. The product was purified with the QIAGEN kit. See patent CN103298955B. The experiment was repeated 10 times. The detection results are shown in Table 2.

TABLE 2
The detected numbers in the case of amplicon library construction using four kinds of primers

| | | | | | | | Primers | Primers with |
|---|---|---|---|---|---|---|---|---|
| | | | | | | Primers with | with G | G added to |
| | | | | Detected | Ordinary | phosphorylated | added to | 5′ end and |
| Reference | GENE | AA | AF | number | primers | 5′ end | 5′ end | phosphorylated |
| HD734 | PIK3CA | H1047R | 30% | 10 | 10 | 10 | 10 | 10 |
| | KRAS | G13D | 25% | 10 | 10 | 10 | 10 | 10 |
| | BRAF | V600E | 8% | 10 | 10 | 10 | 10 | 10 |
| | KRAS | G12S | 1.3% | 10 | 6 | 7 | 9 | 10 |
| | PIK3CA | E542K | 1.3% | 10 | 6 | 8 | 8 | 9 |
| | BRAF | V600M | 1.0% | 10 | 5 | 6 | 8 | 10 |
| | EGFR | T790M | 1.0% | 10 | 4 | 5 | 8 | 10 |
| HD752 | FGFR2 | S252W | 0.00% | 10 | 0 | 0 | 0 | 0 |
| | FLT3 | D835Y | 0.00% | 10 | 0 | 0 | 0 | 0 |
| | GNA11 | Q209L | 0.00% | 10 | 0 | 0 | 0 | 0 |
| | GNAQ | Q209L | 0.00% | 10 | 0 | 0 | 0 | 0 |
| | IDH1 | R132H | 0.00% | 10 | 0 | 0 | 0 | 0 |
| | IDH2 | R140Q | 0.00% | 10 | 0 | 0 | 0 | 0 |
| | JAK2 | V617F | 0.00% | 10 | 0 | 0 | 0 | 0 |
| | KIT | D816V | 0.00% | 10 | 0 | 0 | 0 | 0 |

Results analysis: when testing the wild-type reference, none of the sites was detected using the four kinds of primers. For sites with higher mutation frequency (AF≥8%), all the four kinds of primers were able to be detected in 10 times of detection; for low-frequency sites (1%≤MAF≤1.5%), for 4 low-frequency sites, the detection rate of the ordinary primers was 52.5%, the detection rate of the primers with phosphorylated 5′ end was 65%, and the detection rate of primers with G added to 5′ end was 82.5%, and the detection rate of primers with G added to 5′ end and phosphorylated was 97.5%. It could be seen from the results that, compared to phosphorylation modification, adding G at the 5′ end was more beneficial for low-frequency detection. If phosphorylation modification was performed in addition to adding G, the effect would be better.

### Example 4: Detection of Sites of Commercial cfDNA Reference

Positive reference HD813 and negative reference HD815 (100% Wildtype) from Horizon were used to verify the detection effects of different primers for library construction.

The primer sequences and modifications are shown in Table 17, including 4 kinds of primers, ordinary primers (1), primer (2) that is the ordinary primers with phosphorylated 5′ end, primer (3) that is is the ordinary primers with G added to 5′ end, and primers (4) that is ordinary primers with G added to 5′ end and phosphorylated, the concentration of each primer in each kind of primer pool was 400 nM. A kit (Phusion U multiplex PCR master mix, Thermo Scientific) was used to perform the multiplex PCR reaction in a PCR reaction system as follows. 10 μL of primer mixture was added, 30 ng of template was added, the other components were added following the instructions, and the system was finally made up to 50 μL with water. The reaction conditions were set according to the instructions. The product was purified using a PCR product purification kit (QIAGEN), and eluted in 20 μL. The product obtained using the first primers needed to be phosphorylated. T4 PNK enzyme from NEB was used, and the reaction system and conditions were set according to the instructions of the enzyme. The product was purified with the QIAGEN kit. See patent CN103298955B. The experiment was repeated 10 times. The detection results are shown in Table 3.

TABLE 3
The detected numbers in the case of amplicon library construction using four kinds of primers

| | | | | | | | Primers | Primers with |
|---|---|---|---|---|---|---|---|---|
| | | | | | | Primers with | with G | G added to |
| | | | | Detected | Ordinary | phosphorylated | added to | 5′ end and |
| Reference | GENE | AA | AF | number | primers | 5′ end | 5′ end | phosphorylated |
| HD813 | EGFR | L858R | 1.00% | 10 | 6 | 8 | 8 | 10 |
| | EGFR | AE746-A750 | 1.00% | 10 | 5 | 6 | 9 | 9 |
| | EGFR | T790M | 1.00% | 10 | 5 | 5 | 8 | 10 |
| | EGFR | V769-D770insASV | 1.00% | 10 | 7 | 7 | 7 | 10 |
| | KRAS | G12D | 1.30% | 10 | 6 | 7 | 9 | 10 |
| | NRAS | Q61K | 1.30% | 10 | 5 | 6 | 8 | 9 |
| | NRAS | A59T | 1.30% | 10 | 4 | 5 | 8 | 10 |
| | PIK3CA | ES45K | 1.30% | 10 | 6 | 7 | 8 | 10 |
| HD815 | EGFR | L858R | 0.00% | 10 | 0 | 0 | 0 | 0 |
| | EGFR | ΔE746-A750 | 0.00% | 10 | 0 | 0 | 0 | 0 |
| | EGFR | T790M | 0.00% | 10 | 0 | 0 | 0 | 0 |
| | EGFR | V769-D770insASV | 0.00% | 10 | 0 | 0 | 0 | 0 |
| | KRAS | G12D | 0.00% | 10 | 0 | 0 | 0 | 0 |
| | NRAS | Q61K | 0.00% | 10 | 0 | 0 | 0 | 0 |
| | NRAS | A59T | 0.00% | 10 | 0 | 0 | 0 | 0 |
| | PIK3CA | E545K | 0.00% | 10 | 0 | 0 | 0 | 0 |

Results analysis: when testing the wild-type reference, none of the sites was detected using the four kinds of primers. For sites with 1%≤MAF≤5%, the detection rate of the ordinary primers was 53.8%, the detection rate of the primers with phosphorylated 5′ end was 63.8%, and the detection rate of the primers with G added to 5′ end was 81.3%, and the detection rate of the primers with G added to 5′ end and phosphorylated was 97.5%. Comparing primers 1 and 2, it could be seen that phosphorylation of the 5′ end is beneficial to the detection of low-frequency sites. Comparing primers 2 with primers 3, it could be seen that adding G to the 5′ end is beneficial to the detection of low-frequency sites. By comprehensive comparison, adding G at the 5′ end in combination with phosphorylation was more conducive to low-frequency mutation detection.

### Example 5: Detection of Sites in Self-Made Reference gDNA

Reference A containing multiple mutation sites was formulated by blending multiple cell lines. The mutation frequency of the sites is shown in Table 2. The four kinds of primers in Example 2 were used for detection. The multiplex PCR method was the same as in Example 2. The product obtained using the first primer was phosphorylated using Invitrogen's T4 PNK kit, and purified using the QIAGEN kit. For reactions such as A-addition, adaptor ligation, and library pre-amplification, see patent CN108251515A. The experiment was repeated 10 times. The detection results are shown in Table 4.

Results analysis: when testing the wild-type reference, none of the sites was detected using the four kinds of primers. For sites with 2%≤MAF≤5%, the detection rate of the ordinary primers was 85%, the detection rate of the primers with phosphorylated 5′ end was 88.3%, and the detection rate of the primers with G added to 5′ end was 90%, and the detection rate of the primers with G added to 5′ end and phosphorylated was 100%. For sites with 0.5%≤MAF≤1%, the detection rate of the ordinary primers was 58.6%, the detection rate of the primers with phosphorylated 5′ end was 71.4%, and the detection rate of the primers with G added to 5′ end was 82.1%, and the detection rate of primer 4 was 93.6%. By comprehensive comparison, adding G at the 5′ end in combination with phosphorylation was more conducive to low-frequency mutation detection.

TABLE 4
The detected numbers in the case of amplicon library construction using four kinds of primers

| | | | | | | Primers | Primers with |
|---|---|---|---|---|---|---|---|
| | | | | | Primers with | with G | G added to |
| | | | Detected | Ordinary | phosphorylated | added to | 5′ end and |
| GENE | AA | AF | number | primers | 5′ end | 5′ end | phosphorylated |
| CTNNB1 | p.S45del | 2.68% | 10 | 9 | 9 | 9 | 10 |
| DNAH2 | p.T2172I | 0.63% | 10 | 6 | 8 | 9 | 10 |
| ERBB3 | p.N126K | 0.77% | 10 | 7 | 7 | 9 | 10 |
| FBXW7 | p.R465H | 0.97% | 10 | 6 | 7 | 8 | 9 |
| KRAS | p.G13D | 4.00% | 10 | 9 | 9 | 9 | 10 |
| PIK3CA | p.E545K | 0.98% | 10 | 5 | 6 | 8 | 9 |
| PIK3CA | p.D549N | 0.98% | 10 | 7 | 7 | 8 | 10 |
| PIK3CA | p.R88Q | 0.64% | 10 | 5 | 6 | 8 | 10 |
| PIK3CA | p.G118D | 0.96% | 10 | 8 | 8 | 9 | 9 |
| PPP2R1A | p.R183P | 0.66% | 10 | 5 | 6 | 8 | 10 |
| PTEN | p.R130X | 0.86% | 10 | 8 | 8 | 9 | 9 |
| PTEN | p.R233X | 0.99% | 10 | 8 | 7 | 8 | 9 |
| KRAS | G13D | 0% | 10 | 0 | 0 | 0 | 0 |
| BRAF | V600E | 0% | 10 | 0 | 0 | 0 | 0 |
| KRAS | G12S | 0% | 10 | 0 | 0 | 0 | 0 |
| EGFR | p.T790M | 5.00% | 10 | 9 | 9 | 10 | 10 |
| EGFR | p.T790M | 2.00% | 10 | 8 | 9 | 9 | 10 |
| EGFR | p.T790M | 1.00% | 10 | 6 | 8 | 9 | 10 |
| EGFR | p.T790M | 0.50% | 10 | 4 | 7 | 8 | 7 |
| PIK3CA | p.H1047R | 5.00% | 10 | 9 | 9 | 9 | 10 |
| PIK3CA | p.H1047R | 2.00% | 10 | 7 | 8 | 8 | 10 |
| PIK3CA | p.H1047R | 1.00% | 10 | 5 | 7 | 7 | 10 |
| PIK3CA | p.H1047R | 0.50% | 10 | 2 | 7 | 7 | 9 |

### Example 6: Detection of Sites in Self-Made Reference cfDNA

The extracted cell line DNA was fragmented to about 170 bp by ultrasound. After passing the 2100 quality inspection, reference A containing multiple mutation sites was formulated by blending multiple cell lines. The mutation frequency of each site is shown in Table 2. The four kinds of primers in Example 2 were used for detection. The multiplex PCR method was the same as in Example 2. The product obtained using the first primer was phosphorylated using Invitrogen's T4 PNK kit, and purified using the QIAGEN kit. For reactions such as A-addition, adaptor ligation, and library pre-amplification, see patent CN108251515A. The experiment was repeated 10 times. The detection results are shown in Table 5.

Results analysis: when testing the wild-type reference, none of the sites was detected using the four kinds of primers. For sites with 2%≤MAF≤55%, the detection rate of primer 1 was 52/60=86.7%, the detection rate of primer 2 was 56/60=93.3%, and the detection rate of primer 3 was 95%, and the detection rate of primer 4 was 98.33%. For sites with 0.5%≤MAF≤1%, the detection rate of primer 1 was 49.3%, the detection rate of primer 2 was 57.1%, and the detection rate of primer 3 was 70%, and the detection rate of primer 4 was 84.3%. By comprehensive comparison, primer 4 (with G added at the 5′ end in combination with phosphorylation) is more conducive to low-frequency mutation detection.

TABLE 5
The detected numbers in the case of amplicon library construction using four kinds of primers

| | | | | | | Primers | Primers with |
|---|---|---|---|---|---|---|---|
| | | | | | Primers with | with G | G added to |
| | | | Detected | Ordinary | phosphorylated | added to | 5′ end and |
| GENE | AA | AF | number | primers | 5′ end | 5′ end | phosphorylated |
| CTNNB1 | p.S45del | 2.68% | 10 | 9 | 10 | 10 | 10 |
| DNAH2 | p.T2172I | 0.63% | 10 | 4 | 5 | 6 | 9 |
| ERBB3 | p.N126K | 0.77% | 10 | 4 | 5 | 6 | 8 |
| FBXW7 | p.R465H | 0.97% | 10 | 5 | 6 | 7 | 9 |
| KRAS | p.G13D | 4.00% | 10 | 10 | 10 | 10 | 10 |
| PIK3CA | p.E545K | 0.98% | 10 | 5 | 7 | 8 | 9 |
| PIK3CA | p.D549N | 0.98% | 10 | 4 | 7 | 8 | 9 |
| PIK3CA | p.R88Q | 0.64% | 10 | 4 | 7 | 8 | 2 |
| PIK3CA | p.G118D | 0.96% | 10 | 5 | 6 | 6 | 7 |
| PPP2R1A | p.R183P | 0.66% | 10 | 6 | 7 | 8 | 10 |
| PTEN | p.R130X | 0.86% | 10 | 5 | 8 | 8 | 9 |
| PTEN | p.R233X | 0.99% | 10 | 6 | 8 | 8 | 8 |
| KRAS | G13D | 0% | 10 | 0 | 0 | 0 | 0 |
| BRAF | V600E | 0% | 10 | 0 | 0 | 0 | 0 |
| KRAS | G12S | 0% | 10 | 0 | 0 | 0 | 0 |
| EGFR | p.T790M | 5.00% | 10 | 8 | 9 | 9 | 10 |
| EGFR | p.T790M | 2.00% | 10 | 6 | 8 | 8 | 9 |
| EGFR | p.T790M | 1.00% | 10 | 5 | 5 | 7 | 7 |
| EGFR | p.T790M | 0.50% | 10 | 2 | 3 | 5 | 7 |
| PIK3CA | p.H1047R | 5.00% | 10 | 10 | 10 | 10 | 10 |
| PIK3CA | p.H1047R | 2.00% | 10 | 9 | 9 | 10 | 10 |
| PIK3CA | p.H1047R | 1.00% | 10 | 6 | 7 | 8 | 9 |
| PIK3CA | p.H1047R | 0.50% | 10 | 3 | 4 | 5 | 8 |

### Example 7: Detection of Sites in Nucleosomes

Nucleosomes were prepared using EpiScope® Nucleosome Preparation Kit (Takara Code No. 5333). Reference C containing multiple mutation sites was formulated by blending the nucleosomes prepared from multiple cell lines. The mutation frequency of each site is shown in Table 2. The four kinds of primers in Example 2 were used for detection. The multiplex PCR method was the same as in Example 2. The product obtained using the first primer was phosphorylated using Invitrogen's T4 PNK kit., and purified using the QIAGEN kit. For reactions such as A-addition, adaptor ligation, and library pre-amplification, see patent CN108251515A. The experiment was repeated 10 times. The detection results are shown in Table 6.

Results analysis: when testing the wild-type reference, none of the sites was detected using the four kinds of primers. For sites with 2%≤MAF≤5%, the detection rate of primer 1 was 86.7%, the detection rate of primer 2 was 93.3%, and the detection rate of primer 3 was 95%, and the detection rate of primer 4 was 98.3%. For sites with 0.5%≤MAF≤1% the detection rate of primer 1 was 50.7%, the detection rate of primer 2 was 62.1%, and the detection rate of primer 3 was 75%, and the detection rate of primer 3 was 86.4%. By comprehensive comparison, primer 4 (with G added at the 5′ end in combination with phosphorylation) is more conducive to low-frequency mutation detection.

TABLE 6
The detected numbers in the case of amplicon library construction using four kinds of primers

| | | | | | | Primers | Primers with |
|---|---|---|---|---|---|---|---|
| | | | | | Primers with | with G | G added to |
| | | | Detected | Ordinary | phosphorylated | added to | 5′ end and |
| GENE | AA | AF | number | primer | 5′ end | 5′ end | phosphorylated |
| CTNNB1 | p.S45del | 2.68% | 10 | 9 | 10 | 10 | 10 |
| DNAH2 | p.T2172I | 0.63% | 10 | 4 | 5 | 7 | 8 |
| ERBB3 | p.N126K | 0.77% | 10 | 4 | 5 | 7 | 9 |
| FBXW7 | p.R465H | 0.97% | 10 | 5 | 6 | 7 | 9 |
| KRAS | p.G13D | 4.00% | 10 | 10 | 10 | 10 | 10 |
| PIK3CA | p.E545K | 0.98% | 10 | 6 | 8 | 8 | 10 |
| PIK3CA | p.D549N | 0.98% | 10 | 6 | 7 | 8 | 9 |
| PIK3CA | p.R88Q | 0.64% | 10 | 6 | 7 | 8 | 9 |
| PIK3CA | p.G118D | 0.96% | 10 | 5 | 6 | 9 | 8 |
| PPP2R1A | p.R183P | 0.66% | 10 | 6 | 7 | 8 | 10 |
| PTEN | p.R130X | 0.86% | 10 | 5 | 8 | 8 | 9 |
| PTEN | p.R233X | 0.99% | 10 | 7 | 8 | 8 | 8 |
| KRAS | G13D | 0% | 10 | 0 | 0 | 0 | 0 |
| BRAF | V600E | 0% | 10 | 0 | 0 | 0 | 0 |
| KRAS | G12S | 0% | 10 | 0 | 0 | 0 | 0 |
| EGFR | p.T790M | 5.00% | 10 | 7 | 9 | 9 | 10 |
| EGFR | p.T790M | 2.00% | 10 | 6 | 8 | 8 | 10 |
| EGFR | p.T790M | 1.00% | 10 | 5 | 5 | 7 | 7 |
| EGFR | p.T790M | 0.50% | 10 | 3 | 4 | 5 | 8 |
| PIK3CA | p.H1047R | 5.00% | 10 | 10 | 10 | 10 | 10 |
| PIK3CA | p.H1047R | 2.00% | 10 | 9 | 9 | 10 | 10 |
| PIK3CA | p.H1047R | 1.00% | 10 | 6 | 7 | 9 | 9 |
| PIK3CA | p.H1047R | 0.50% | 10 | 3 | 4 | 7 | 8 |

### Example 8: Detection of Sites in Intestinal Cancer Plasma Samples

21 plasma samples from intestinal cancer and 10 plasma samples from healthy humans were collected. DNA was extracted and subjected to multiplex PCR using the four kinds of primers in Table 17. The multiplex PCR amplification was performed using Vazyme multiplex enzymes, and the PCR products were subjected to experiments such as A-addition, adaptor ligation, and library pre-amplification using the KAPA library construction kit. The detection results are shown in Table 7.

TABLE 7
Detection of sites in intestinal cancer samples

| | | | | | Primers with G |
|---|---|---|---|---|---|
| | Sample | | Primers with | Primers with G | added to 5′ end |
| Sample type | No. | Ordinary primer | phosphorylated 5′ end | added to 5′ end | and phosphorylated |
| Intestinal cancer | 1 | / | / | KRAS p.G13D 0.3% | KRAS p.G13D 0.3% |
| Intestinal cancer | 2 | / | / | / | / |
| Intestinal cancer | 3 | KRAS p.Q61H 0.3% | KRAS p.Q61H 0.4% | KRAS p.Q61H 1.1% | KRAS p.Q61H 1.4% |
| Intestinal cancer | 4 | KRAS p.G12D 0.5% | KRAS p.G12D 0.5% | KRAS p.G12D 1.7% | KRAS p.G12D 1.9% |
| Intestinal cancer | 5 | KRAS p.G13D 0.4% | KRAS p.G13D 0.6% | KRAS p.G13D 0.9% | KRAS p.G13D 1.2% |
| Intestinal cancer | 6 | / | / | BRAF p.V600E 0.4% | BRAF p.V600E 0.5% |
| | | | | | TP53 p.R273C 0.3% |
| Intestinal cancer | 7 | KRAS p.G12D 0.2% | KRAS p.G12D 0.5% | KRAS p.G12D 1.2% | KRAS p.G12D 1.3% |
| Intestinal cancer | 8 | / | / | KRAS p.G12C 0.3% | KRAS p.G12C 0.3% |
| Intestinal cancer | 9 | / | / | / | / |
| Intestinal cancer | 10 | BRAF p.V600E 0.7% | BRAF p.V600E 0.8% | BRAF p.V600E 2.1% | BRAF p.V600E 2.5% |
| | | | | | TP53 p.R273C 0.4% |
| Intestinal cancer | 11 | / | / | / | / |
| Intestinal cancer | 12 | / | / | KRAS p.G12V 0.3% | KRAS p.G12V 0.4% |
| | | | | APC p.R1450* 0.2% | APC p.R1450* 0.2% |
| Intestinal cancer | 13 | BRAF p.G12D 0.3% | BRAF p.G12D 0.4% | BRAF p.G12D 1.1% | BRAF p.G12D 1.3% |
| | | | | | TP53 p.R282W 0.3% |
| Intestinal cancer | 14 | / | / | KRAS p.G12S 0.3% | KRAS p.G12S 0.4% |
| Intestinal cancer | 15 | / | KRAS p.G12C 0.4% | KRAS p.G12C 1% | KRAS p.G12C 1.2% |
| Intestinal cancer | 16 | KRAS p.G12R 0.9% | KRAS p.G12R 1.2% | KRAS p.G12R 1.8% | KRAS p.G12R 2% |
| | | | | TP53 p.R273H 0.3% | TP53 p.R273H 0.5% |
| Intestinal cancer | 17 | / | / | / | / |
| Intestinal cancer | 18 | / | / | / | / |
| Intestinal cancer | 19 | / | / | / | BRAF p.V600E 0.4% |
| Intestinal cancer | 20 | / | / | KRAS p.G12D 0.4% | KRAS p.G12D 0.4% |
| | | | | | APC p.R876* 0.3% |
| Intestinal cancer | 21 | KRAS p.G12V 0.6% | KRAS p.G12V 0.8% | KRAS p.G12V 1.7% | KRAS p.G12V 1.8% |
| Healthy person | 22 | / | / | | / |
| Healthy person | 23 | / | / | | / |
| Healthy person | 24 | / | / | | / |
| Healthy person | 25 | / | / | | / |
| Healthy person | 26 | / | / | | / |
| Healthy person | 27 | / | / | | / |
| Healthy person | 28 | / | / | | / |
| Healthy person | 29 | / | / | | / |
| Healthy person | 30 | / | / | | / |
| Healthy person | 31 | / | / | | / |

All healthy humans presented negative results in the detection using the 4 kinds of primers. For the CRC detection, it was detected in 8 samples in the case where amplicon library construction was performed using the ordinary primers (single-site detection for all) with a detection rate of 38.1%, in 9 samples when using the primers with phosphorylated 5′ end with the detection rate of 42.9% (single-site detection for all), in 15 samples using the primers with G added to 5′ end with a detection rate of 71.4% (double-site detection for 2 samples), and in 16 samples when using the primers with G added to 5′ end and phosphorylated with a detection rate of 76.2% (double-site detection for 6 samples). It could be seen that adding G to the 5′ end of the primer significantly improved the detection rate. If G was added in combination with phosphorylation, the detection would be more stable.

### Table 9 Detection of Sites in Cervical Exfoliated Cell Samples

Cervical exfoliated cell samples with clear pathological information were collected, including 15 cases of cervical cancer, 5 cases of CIN3, 5 cases of CIN2, and 5 cases of CIN1, and then multiplex PCR was performed on these 30 samples using the 4 kinds of primers in Table 17. The multiplex PCR amplification was performed using Vazyme multiplex enzymes, and the PCR products were subjected to experiments such as A-addition, adaptor ligation, and library pre-amplification using the NEB library construction kit. The detection results are shown in Table 8.

TABLE 8
Detection of sites in cervical exfoliated cell samples

| | | | | | Primers with G added |
|---|---|---|---|---|---|
| | Sample | | Primers with | Primers with G added | to 5′ end and |
| Sample type | No. | Ordinary primer | phosphorylated 5′ end | to 5′ end | phosphorylated |
| Cervical | 1 | PIK3CA p.E545K 17.2% | PIK3CA p.E545K 18.9% | PIK3CA p.E545K 17.4% | PIK3CA p.E545K 16.3% |
| cancer | | | PIK3CA p.E726K 1.3% | PIK3CA p.E453K 1.4% | PIK3CA p.E453K 1.4% |
| | | | | | FBXW7 p.R479G 1.3% |
| Cervical | 2 | / | / | PIK3CA p.E726K 1.3% | PIK3CA p.E726K 1.7% |
| cancer | | | | | |
| Cervical | 3 | PIK3CA p.E545K 16..3% | PIK3CA p.E545K 17.3% | PIK3CA p.E545K 17.4% | PIK3CA p.E545K 17.4% |
| cancer | | | | | |
| Cervical | 4 | ERBB2 p.S310Y 21.5% | ERBB2 p.S310Y 22.5% | ERBB2 p.S310Y 21.2% | ERBB2 p.S310Y 22.6% |
| cancer | | | | | |
| Cervical | 5 | PIK3CA p.E545K 18.9% | PIK3CA p.E545K 20.6% | PIK3CA p.E545K 20.1% | PIK3CA p.E453K 1.5% |
| cancer | | | FBXW7 p.R479G 2.7% | FBXW7 p.R479G 1.3% | PIK3CA p.E545K 20.4% |
| | | | | | FBXW7 p.R479G 1.6% |
| Cervical | 6 | / | / | PIK3CA p.E726K 1.6% | PIK3CA p.E726K 1.6% |
| cancer | | | | | |
| Cervical | 7 | PIK3CA p.H1047R 17.2% | PIK3CA p.H1047R 18.5% | PIK3CA p.H1047R 18.1% | PIK3CA p.H1047R 19.1% |
| cancer | | | | | |
| Cervical | 8 | PIK3CA p.E542K 51% | PIK3CA p.E542K 53% | PIK3CA p.E542K 50.6% | PIK3CA p.E542K 52.6% |
| cancer | | | | | |
| Cervical | 9 | / | PIK3CA p.K111del 4.8% | PIK3CA p.K111del 3.2% | PIK3CA p.K111del 3.2% |
| cancer | | | PTEN p.G165E 2.2% | PTEN p.G165E 2.2% | PTEN p.G165E 2.2% |
| Cervical | 10 | PIK3CA p.E726K 2.2% | PIK3CA p.E726K 2.5% | PIK3CA p.E726K 2.5% | PIK3CA p.E726K 2.5% |
| cancer | | | | | |
| Cervical | 11 | CDKN2A p.W110X 16.9% | CDKN2A p.W110X 18.9% | CDKN2A p.W110X 20.9% | CDKN2A p.W110X 20.9% |
| cancer | | | | | |
| Cervical | 12 | / | / | PIK3CA p.E542K 1.9% | PIK3CA p.E542K 1.9% |
| cancer | | | | | |
| Cervical | 13 | / | / | | PIK3CA p.E545K 1.4% |
| cancer | | | | | |
| Cervical | 14 | / | | FBXW7 p.R479G 2.7% | FBXW7 p.R479G 2.7% |
| cancer | | | | | |
| Cervical | 15 | PIK3CA p.E545K 6.5% | PIK3CA p.E545K 7.2% | PIK3CA p.E545K 8.8% | PIK3CA p.E545K 8.6% |
| cancer | | | PIK3CA p.E726K 1.2% | PIK3CA p.E726K 1.3% | PIK3CA p.E726K 1.5% |
| CIN3 | 16 | / | / | PIK3CA p.N107S 1.5% | PIK3CA p.N107S 1.2% |
| CIN3 | 17 | / | PIK3CA p.E542K 6.5%; | PIK3CA p.E542K 6.8%; | PIK3CA p.E542K 6.6%; |
| | | | PIK3CA p.E545K 9.9%; | PIK3CA p.E545K 8.4%; | PIK3CA p.E545K 9.4%; |
| | | | | PIK3CA p.N107S 1.3% | PIK3CA p.N107S 1.2% |
| CIN3 | 18 | / | / | / | PIK3CA p.E545K 1.4%; |
| CIN3 | 19 | / | PIK3CA p.E542K 7.9% | PIK3CA p.E542K 7.6% | PIK3CA p.E542K 7.4% |
| | | | PIK3CA p.E545K 9% | PIK3CA p.E545K 8.9% | PIK3CA p.E545K 8.8% |
| CIN3 | 20 | / | / | / | / |
| CIN2 | 21 | / | PIK3CA p.G118D 12.1% | PIK3CA p.G118D 11.8% | PIK3CA p.G118D 11.1% |
| CIN2 | 22 | / | / | / | / |
| CIN2 | 23 | / | / | / | / |
| CIN2 | 24 | / | / | PIK3CA p.E542K 1.8% | PIK3CA p.E542K 1.4% |
| CIN2 | 25 | / | / | / | PIK3CA p.E542K 1.4% |
| CIN1 | 26 | / | / | / | / |
| CIN1 | 27 | / | / | / | / |
| CIN1 | 28 | / | / | / | / |
| CIN1 | 29 | / | / | / | / |
| CIN1 | 30 | / | / | / | / |

In the library of cervical cancer samples, the detection rate of primer 1 was 9/15=60%, and the detection rate of cervical cancer in the case where the library was constructed with primer 2 was 10/15=66.7%. The detection rate of primer 3 was 13/15=86.7%, and the detection rate of cervical cancer in the case where the library was constructed with primer 4 was 15/15=100%. In CIN3, the detection rate of primer 1 was 0/5=0%, the detection rate of primer 2 was 2/5=40%; the detection rate of primer 3 was 3/5=60%, and the detection rate of primer 4 was 4/5=80%. In CIN2, the detection rate of primer 1 was 0/5=0%, the detection rate of primer 2 was 1/5=20%; the detection rate in the library constructed with primer 3 was 2/5=40%, and the detection rate of primer 4 was 3/5=60%. No sites were detected in the CIN1 sample using the library constructed with four kinds of primers. Primer 4 (with G added at the 5′ end in combination with phosphorylation) could detect more chromosomal abnormalities.

### Example 10: Detection of Sites in Urine Samples

20 urine samples from urothelial cancer and 10 urine samples from healthy humans were collected. DNA was extracted and subjected to multiplex PCR using the four kinds of primers. Thermo Fisher reagents were used in multiplex PCR, and the PCR products were subjected to experiments such as A-addition, adaptor ligation, and library amplification using the Vazyme library construction kit. The detection results are shown in Table 9.

TABLE 9
Detection of sites in urine samples

| | Sample | | Primers with | Primers with G | Primers with G added to |
|---|---|---|---|---|---|
| Sample type | No. | Ordinary primer | phosphorylated 5′ end | added to 5′ end | 5′ end and phosphorylated |
| Urothelial | 1 | KRAS c.735G > T 3.1% | TERT c.54C > A 2.5% | TERT c.54C > A 2.1% | TERT c.54C > A 2.3% |
| cancer | | | KRAS c.735G > T 3.4% | KRAS c.735G > T 3.9% | KRAS c.735G > T 4.1% |
| Urothelial | 2 | / | FGFR3 c.742C > T 3.2% | FGFR3 c.742C > T 3.5% | FGFR3 c.742C > T 3.5% |
| cancer | | | KRAS c.57G > C 4.1% | KRAS c.57G > C 4.3% | HRAS c.182A > T 1.2% |
| | | | | | KRAS c.57G > C 4.3% |
| Urothelial | 3 | / | / | / | / |
| cancer | | | | | |
| Urothelial | 4 | KRAS c.76A > T 4.1% | KRAS c.76A > T 4.5% | KRAS c.76A > T 4.5% | KRAS c.76A > T 4.1% |
| cancer | | | PIK3CA c.278G > A 1.4% | PIK3CA c.278G > A 1.4% | ERBB2 c.308G > A 1.3% |
| | | | | | PIK3CA c.278G > A 1.5% |
| Urothelial | 5 | / | / | / | FGFR3 c.1111A > T 1.5% |
| cancer | | | | | |
| Urothelial | 6 | ERBB2 c.914C > G 4.2% | ERBB2 c.914C > G 4.1% | ERBB2 c.914C > G 4.1% | ERBB2 c.914C > G 3.9% |
| cancer | | | | | |
| Urothelial | 7 | / | / | / | HRAS c.38G > A 0.98% |
| cancer | | | | | |
| Urothelial | 8 | / | | TERT c.93T > G 1.9% | TERT c.93T > G 2.3% |
| cancer | | | | TERT c.124C > A 2.5% | TERT c.124C > A 2.3% |
| Urothelial | 9 | | KRAS c.38G > A 3.1% | KRAS c.38G > A 3.5% | KRAS c.38G > A 4.1% |
| cancer | | | TERT c.80C > T 2.1% | TERT c.80C > T 2.5% | TERT c.80C > T 2.3% |
| Urothelial | 10 | FGFR3 c.746C > G 3.6% | FGFR3 c.746C > G 3.6% | FGFR3 c.746C > G 3.2% | FGFR3 c.746C > G 3.5% |
| cancer | | ERBB2 c.291G > C 3.2% | ERBB2 c.291G > C 3.4% | ERBB2 c.291G > C 3.1% | ERBB2 c.291G > C 3.9% |
| Urothelial | 11 | / | / | HRAS c.19G > C 3.3% | HRAS c.19G > C 5.2% |
| cancer | | | | | |
| Urothelial | 12 | PIK3CA c.317G > T 5.8% | PIK3CA c.317G > T 5.8% | PIK3CA c.317G > T 5.4% | PIK3CA c.317G > T 6.4% |
| cancer | | | | | |
| Urothelial | 13 | / | / | / | HRAS c.37G > C 1.2% |
| cancer | | | | | ERBB2 c.829G > T 1.9% |
| Urothelial | 14 | PIK3CA c.316G > C 5.2% | PIK3CA c.316G > C 5.2% | PIK3CA c.316G > C 5.1% | PIK3CA c.316G > C 5.4% |
| cancer | | | | | FGFR3 c.1102G > T 1.5% |
| Urothelial | 15 | PIK3CA c.1357G > A 5.5% | PIK3CA c.1357G > A 5.8% | PIK3CA c.1357G > A 5.4% | PIK3CA c.1357G > A 6.1% |
| cancer | | ERBB2 c.1979G > A 2.9% | ERBB2 c.1979G > A 2.1% | ERBB2 c.1979G > A 2.3% | ERBB2 c.1979G > A 3.1% |
| Urothelial | 16 | / | / | / | / |
| cancer | | | | | |
| Urothelial | 17 | / | / | PIK3CA c.323G > A 4.5% | PIK3CA c.323G > A 4.7% |
| cancer | | | | HRAS c.218G > A 1.6% | HRAS c.218G > A 1.2% |
| Urothelial | 18 | / | / | / | / |
| cancer | | | | | |
| Urothelial | 19 | / | / | / | / |
| cancer | | | | | |
| Urothelial | 20 | / | HRAS c.34G > A 3.5% | HRAS c.34G > A 3.2% | HRAS c.34G > A 3.5% |
| cancer | | | | FGFR3 c.749C > A 0.5% | FGFR3 c.749C > A 1.3% |
| Healthy | 21 | / | / | / | / |
| person | | | | | |
| Healthy | 22 | / | / | / | / |
| person | | | | | |
| Healthy | 23 | / | / | / | / |
| person | | | | | |
| Healthy | 24 | / | / | / | / |
| person | | | | | |
| Healthy | 25 | / | / | / | / |
| person | | | | | |
| Healthy | 26 | / | / | / | / |
| person | | | | | |
| Healthy | 27 | / | / | / | / |
| person | | | | | |
| Healthy | 28 | / | / | / | / |
| person | | | | | |
| Healthy | 29 | / | / | / | / |
| person | | | | | |
| Healthy | 30 | / | / | / | / |
| person | | | | | |

All healthy humans presented negative results in the detection. For the detection in urothelial cancer samples, the detection rate in the case where amplicon library construction was performed using the ordinary primer was 7/20=35%, and the detection rate in the case where amplicon library construction was performed using the primers with phosphorylated 5′ end was 10/20=50%, the detection rate in the case where amplicon library construction was performed using the primers with G added to 5′ end was 13/20=65%, the detection rate in the case where amplicon library construction was performed using the primers with G added to 5′ end and phosphorylated was 16/20=80%, showing the best detection effect.

### Example 11: Ploidy Detection Limit

The positive cell line reference hela cells with 6 copies of chromosome 5 verified by FISH were blended with the negative cell line with 2 copies in different proportions to obtain references with different copy numbers. See Table 10 for details. The four kinds of primers in Table 18 were used for amplification respectively, and multiplex high-fidelity enzyme from Thermo Fisher was used for amplification. For the experiments of A-addition, adaptor ligation, and library pre-amplification of the amplicons, see patent CN10329895513. The detection results are shown in Table 10.

TABLE 10
Ploidy detection limit

| | | Theoretical | | | | | | |
|---|---|---|---|---|---|---|---|---|
| | Blending | copy | Duplicate 1 | | | Duplicate 2 | | |
| | ratio | number | GAIN | LOSS | AI | GAIN | LOSS | AI |
| Ordinary | PC | 6 | ✓ | x | x | ✓ | x | x |
| primer | NC | 2 | x | x | x | x | x | x |
| | 50% | 4 | ✓ | x | x | ✓ | x | x |
| | 20% | 2.8 | ✓ | x | x | ✓ | x | x |
| | 10% | 2.4 | x | x | ✓ | x | x | x |
| | 5% | 2.2 | x | x | x | x | x | x |
| | 1% | 2.04 | x | x | x | x | x | x |
| Primers with | PC | 6 | ✓ | x | x | ✓ | x | x |
| phosphorylated | NC | 2 | x | x | x | x | x | x |
| 5′ end | 50% | 4 | ✓ | x | x | ✓ | x | x |
| | 20% | 2.8 | ✓ | x | x | ✓ | x | x |
| | 10% | 2.4 | x | x | ✓ | x | x | ✓ |
| | 5% | 2.2 | x | x | ✓ | x | x | x |
| | 1% | 2.04 | x | x | x | x | x | x |
| Primers with | PC | 6 | ✓ | x | x | ✓ | x | x |
| G added to | NC | 2 | x | x | x | x | x | x |
| 5′ end | 50% | 4 | ✓ | x | x | ✓ | x | x |
| | 20% | 2.8 | ✓ | x | x | ✓ | x | x |
| | 10% | 2.4 | x | x | ✓ | x | x | ✓ |
| | 5% | 2.2 | x | x | ✓ | x | x | ✓ |
| | 1% | 2.04 | x | x | x | x | x | x |
| Primers with | PC | 6 | ✓ | x | x | ✓ | x | x |
| G added to | NC | 2 | x | x | x | x | x | x |
| 5′ end and | 50% | 4 | ✓ | x | x | ✓ | x | x |
| phosphorylated | 20% | 2.8 | ✓ | x | x | ✓ | x | x |
| | 10% | 2.4 | ✓ | x | x | ✓ | x | x |
| | 5% | 2.2 | ✓ | x | x | ✓ | x | x |
| | 1% | 2.04 | x | x | ✓ | x | x | ✓ |

The results of tests in duplicate showed that: for amplification with the ordinary primers, ploidy could be stably detected with the lowest detection limit of of 2.8 copies; the lowest detection limit for the primers with phosphorylated 5′ end was 2.4 copies; for the amplification with the primers with G added to 5′ end, the lowest detection limit was 2.2 copies; for amplification with the primers with G added to 5′ end and phosphorylated, the lowest detection limit was 2.04 copies, showing the best detection effect (√ indicated that chromosome 5 was detected as positive, x indicated that chromosome 5 was not detected).

### Example 12: Detection of Ploidy in Intestinal Cancer FFPE Samples

20 FFPE samples of intestinal cancer with clear pathological information were collected. Amplification was performed using the four kinds of primers in Table 18. KAPA high-fidelity enzyme was used in PCR, and the products were subjected to A-addition, adaptor ligation, and library pre-amplification experiments using the KAPA library construction kit. Chromosomal abnormalities were investigated. The results are shown in Table 11.

TABLE 11
Detection of ploidy in intestinal cancer FFPE samples

| | | | | | | | | | | | Primer 4: Primers with | | |
|---|---|---|---|---|---|---|---|---|---|---|---|---|---|
| Sam- | Sam- | | | | Primer 2: Primers with | | | Primer 3: Primers with | | | G added to 5′ end and | | |
| ple | ple | Primer 1: Ordinary primer | | | phosphorylated 5′ end | | | G added to 5′ end | | | phosphorylated | | |
| type | No. | GAIN | LOSS | AI | GAIN | LOSS | AI | GAIN | LOSS | AI | GAIN | LOSS | AI |
| Intes- | 1 | / | / | / | / | / | / | / | / | / | 1q, | / | / |
| tinal | | | | | | | | | | | | | |
| cancer | | | | | | | | | | | | | |
| Intes- | 2 | / | / | / | / | 20p | / | / | 20p | 6q, 11q | 7q | 20p | 6q, 11q |
| tinal | | | | | | | | | | | | | |
| cancer | | | | | | | | | | | | | |
| Intes- | 3 | / | / | / | / | / | / | / | / | / | / | / | 1p,, 5q, |
| tinal | | | | | | | | | | | | | 8p, 8q, |
| cancer | | | | | | | | | | | | | 11p, 14q, |
| | | | | | | | | | | | | | 18p, |
| Intes- | 4 | 1q, 7p, | / | 1p, 3q | 1q, 7p, | / | 1p, 3q, | 1q, 7p, | / | 1p, 3q, | 1q, 7p, | / | 1p, 3q, |
| tinal | | 13q, | | | 7q, 13q, | | 5p, 5q, | 13q, 20q | | 11p, 11q, | 7q, 13q, | | 5p, 5q, |
| cancer | | | | | 20q | | 8p, 8q, | | | 14q, 18p, | 20q | | 8p, 8q, |
| | | | | | | | 11p, 11q, | | | 18q, 22q | | | 11p, 11q, |
| | | | | | | | 18q, 22q | | | | | | 14q, 18p, |
| | | | | | | | | | | | | | 18q, 22q |
| Intes- | 5 | / | / | 3q, 5p, | / | / | 1p, 3q, | / | / | 1p, 3q, | / | / | 1p, 3q, |
| tinal | | | | 8p, 8q, | | | 5p, 5q, | | | 5p, 5q, | | | 5p, 5q, |
| cancer | | | | | | | 8p, 8q, | | | 8p, 8q, | | | 8p, 8q |
| Intes- | 6 | / | / | / | / | 20p | / | / | 20p | / | / | 20p | / |
| tinal | | | | | | | | | | | | | |
| cancer | | | | | | | | | | | | | |
| Intes- | 7 | / | / | 1p, 3q, | 7q | / | 1p, 3q, | / | / | 1p, 3q, | 7q | / | 1p, 3q, |
| tinal | | | | 5p, 5q, | | | 5p, 5q, | | | 5p, 5q, | | | 5p, 5q, |
| cancer | | | | 8p, | | | 8p, 8q18q, | | | 8p, 8q,, | | | 8p, 8q,, |
| | | | | | | | 22q | | | 18q, | | | 18q, 22q |
| Intes- | 8 | / | / | / | / | / | / | / | / | / | 1q, 20q | / | / |
| tinal | | | | | | | | | | | | | |
| cancer | | | | | | | | | | | | | |
| Intes- | 9 | 7q | / | 11p, 11q, | 7q | 20p | 1p, 3q, | 7q | / | 1p, 3q, | 7q | 20p | 1p, 3q, |
| tinal | | | | 18p, | | | 5p, 11p, | | | 5p, 5q, | | | 5p, 5q, |
| cancer | | | | | | | 11q, 14q, | | | 8p, 8q, | | | 8p, 8q, |
| | | | | | | | 18p, | | | 11p, 11q, | | | 11p, 11q, |
| | | | | | | | | | | 18p, | | | 14q, 18p, |
| Intes- | 10 | / | / | / | / | / | / | / | / | / | / | / | / |
| tinal | | | | | | | | | | | | | |
| cancer | | | | | | | | | | | | | |
| Intes- | 11 | 7p, 20q | / | 3q, 5p, | 7p, 20q | / | 1p, 3q, | 7p, 20q | / | 1p, 3q, | 7p, 20q | / | 1p, 3q, |
| tinal | | | | 8p, 8q, | | | 5q, 8p, | | | 5q, 8p, | | | 5q, 8p, |
| cancer | | | | | | | 8q, 11q, | | | 8q, 11q, | | | 8q, 11q, |
| | | | | | | | 14q | | | 14q, 18p, | | | 14q, 18p, |
| Intes- | 12 | / | / | 1p, 3q, | / | / | 1p, 3q, | / | / | 1p, 3q, | / | / | 1p, 3q, |
| tinal | | | | 5p, 8q, | | | 5p, 8p, | | | 5p, 5q, | | | 5p, 5q, |
| cancer | | | | 11p, 18p, | | | 11p, 18p, | | | 8p, 8q, | | | 8p, 8q, |
| | | | | | | | | | | 11p, 18p, | | | 11p, 18p, |
| Intes- | 13 | / | / | / | / | / | / | / | / | / | / | / | 1p, 3q, |
| tinal | | | | | | | | | | | | | 5p, 5q, |
| cancer | | | | | | | | | | | | | 8p, 8q |
| Intes- | 14 | 1q, 7p | / | 6q, 11q, | 1q, 7p | / | 18q, 22q | 1q, 7p | / | 6q, 11q, | 1q, 7p | / | 6q, 11q, |
| tinal | | | | 22q | | | | | | 22q | | | 18q, 22q |
| cancer | | | | | | | | | | | | | |
| Intes- | 15 | / | / | / | / | / | / | / | / | / | / | / | / |
| tinal | | | | | | | | | | | | | |
| cancer | | | | | | | | | | | | | |
| Intes- | 16 | 13q | / | 1p,, 5q, | 1q, 13q | / | 1p,, 5q, | 13q | / | 1p,, 5q, | 1q, 13q | / | 1p,, 5q, |
| tinal | | | | 18p, | | | 8p, 14q, | | | 11p, 14q, | | | 8p, 8q, |
| cancer | | | | | | | 18p, | | | 18p, | | | 11p, 14q, |
| | | | | | | | | | | | | | 18p, |
| Intes- | 17 | / | / | / | / | / | / | / | / | / | / | / | / |
| tinal | | | | | | | | | | | | | |
| cancer | | | | | | | | | | | | | |
| Intes- | 18 | / | / | / | 7q, 13q | / | / | 7q, | / | / | 7q, 13q | / | / |
| tinal | | | | | | | | | | | | | |
| cancer | | | | | | | | | | | | | |
| Intes- | 19 | 13q, 20q | / | 5p, 11q, | 13q, 20q | / | 5p, 5q, | 13q, 20q | / | 5p, 11p, | 13q, 20q | / | 5p, 5q, |
| tinal | | | | 14q, 18p | | | 11q, 14q, | | | 11q, 14q | | | 8p, 8q, |
| cancer | | | | | | | 18p | | | 18p | | | 11p, 11q, |
| | | | | | | | | | | | | | 14q, 18p |
| Intes- | 20 | / | / | 3q, 8p, | / | / | 3q, 8p, | / | / | 3q, 8p, | / | / | 3q, 8p, |
| tinal | | | | 8q, 11p, | | | 8q, 11p, | | | 8q, 11p, | | | 8q, 11p, |
| cancer | | | | 14q | | | 14q, 18p, | | | 14q | | | 14q, 18p, |
| Intes- | 21 | 1q, 20q | / | 1p, 3q, | 1q, 7p, | / | 1p, 3q, | 1q, 7p, | / | 1p, 5p, | 19, 7p, | / | 1p, 3q, |
| tinal | | | | 5p, 11q | 13q, 20q | | 5p, 11q, | 20q | | 11q, 14q, | 13q, 20q | | 5p, 11q, |
| cancer | | | | | | | 14q, | | | 18p | | | 14q, 18p, |
| Intes- | 22 | / | / | / | / | / | / | / | / | 6q, 11q, | 13q | / | / |
| tinal | | | | | | | | | | 22q | | | |
| cancer | | | | | | | | | | | | | |
| Intes- | 23 | / | / | / | / | / | / | / | / | / | 13q, 20q | / | / |
| tinal | | | | | | | | | | | | | |
| cancer | | | | | | | | | | | | | |
| Intes- | 24 | / | / | / | / | / | / | 1q, 13q, | / | / | 1q, 13q, | / | / |
| tinal | | | | | | | | 20q | | | 20q | | |
| cancer | | | | | | | | | | | | | |
| Intes- | 25 | / | / | / | / | / | / | / | 20p | / | / | 20p | / |
| tinal | | | | | | | | | | | | | |
| cancer | | | | | | | | | | | | | |

In the detection of intestinal cancer FFPE samples, the detection rate of chromosome ploidy changes in the case where amplicon library construction was performed using primer 1 was 11/25=44%; the detection rate of chromosome ploidy changes in the case where amplicon library construction was performed using primer 2 was 14/25=56%; the detection rate of chromosome ploidy changes in the case where amplicon library construction was performed using primer 3 was 17/25=68%; the detection rate of chromosome ploidy changes in the case where amplicon library construction was performed using primer 4 was 22/25=88%; and more chromosomal abnormalities could be detected in the case where amplicon library construction was performed using primer 4.

### Example 13 Detection of Ploidy in Cervical Exfoliated Cell Samples

Cervical exfoliated cell samples with clear pathological information were collected, including 15 cases of cervical cancer, 5 cases of CIN3, 5 cases of CIN2, and 5 cases of CIN1. These 30 samples were subjected to different methods to construct a ploidy library. The four kinds of primers in Sequence Listing 2 were used. Amplification was performed by QIAGEN multiplex enzyme (no need to add A to the product), and KAPA library construction kit was used for adaptor ligation and library pre-amplification. See Table 12 for detection results.

TABLE 12
Detection of ploidy in cervical exfoliated cell samples

| | | | | | Primer 2: Primers with | | | Primer 3: Primers with G | | | Primer 4: Primers with G added | | |
|---|---|---|---|---|---|---|---|---|---|---|---|---|---|
| Sample | Sample | Primer 1: Ordinary primer | | | phosphorylated 5′ end | | | added to 5′ end | | | to 5′ end and phosphorylated | | |
| type | No. | GAIN | LOSS | AI | GAIN | LOSS | AI | GAIN | LOSS | AI | GAIN | LOSS | AI |
| Cervical | 1 | 1q, 16q | / | 16p | 1q, 5p, | / | 3q, 9p, | 1q, 5p, | / | 3q, 16p | 1q, 5p, | / | 3q, 9p, |
| cancer | | | | | 9q, 16q | | 16p | 9q, 16q | | | 9q, 16q | | 16p |
| Cervical | 2 | / | / | 16p | 1q, 16q | / | 3q, 9p, | 1q, 9q, | / | 3q, 9p, | 1q, 5p, | / | 34, 9p, |
| cancer | | | | | | | 16p | 16q | | 16p | 9q, 16q | | 16p |
| Cervical | 3 | 34, 9p | 3p | / | 14, 3q, | / | 4q | 1q, 3q, | 3p, 4p | 4q | 1q, 3q, | 3p, 4p | 4q |
| cancer | | | | | 9p | | | 9p | | | 9p | | |
| Cervical | 4 | / | / | / | / | / | / | / | / | 2q, 3q, | 2p, 5p | 5q | 2q, 3q, |
| cancer | | | | | | | | | | 10p, 10q, | | | 4q, 6p, |
| | | | | | | | | | | 11p, 13q | | | 6q, 7p, |
| | | | | | | | | | | | | | 7q, 8q, |
| | | | | | | | | | | | | | 10p, 10q, |
| | | | | | | | | | | | | | 11p, 13q |
| Cervical | 5 | / | / | 4q, 5p, | 3q | / | 4p, 7p, | 3q | / | 4q, 5p, | 3q | 3p | 4p, 4q, |
| cancer | | | | 11p, 13q | | | 11p, 13q | | | 11p, 13q | | | 5p, 5q, |
| | | | | | | | | | | | | | 7p, 11p, |
| | | | | | | | | | | | | | 13q |
| Cervical | 6 | / | / | 18q | 3q, 8q | / | 18q | 3q, 8q | / | 18q | 3q, 8q | / | 18q |
| cancer | | | | | | | | | | | | | |
| Cervical | 7 | / | / | / | / | / | / | / | / | / | / | 4p, 4q | / |
| cancer | | | | | | | | | | | | | |
| Cervical | 8 | / | / | / | / | / | / | 3q | / | 4p, 5q, | 3q | / | 4p, 5q, |
| cancer | | | | | | | | | | 8q | | | 8q |
| Cervical | 9 | / | / | / | / | / | 3q, 4p, | / | / | 3q, 4p, | / | / | 3q, 4p, |
| cancer | | | | | | | 5q, 6p, | | | 18p | | | 5q, 6p, |
| | | | | | | | 8p, 8q, | | | | | | 8p, 8q, |
| | | | | | | | 18p | | | | | | 18p |
| Cervical | 10 | / | / | / | / | / | / | 1p, 3q | / | 4q, 17q | 1p, 1q, | / | 4q, 17q |
| cancer | | | | | | | | | | | 2p, 3q | | |
| Cervical | 11 | / | / | / | 1p, 1q, | / | 4q, 16q, | 1p, 1q, | / | 4q, 16q | 1p, 1q, | / | 4q, 16q, |
| cancer | | | | | 2p, 3q | | 17q | 2p | | | 2p, 3q | | 17q |
| Cervical | 12 | / | / | / | / | / | / | / | / | / | / | / | 3q, 7p, |
| cancer | | | | | | | | | | | | | 11p |
| Cervical | 13 | 1q, 3q, | 2q, 4q | 4p, 9q, | 1q, 3q, | 2q, 4q | 4p, 9q, | 1q, 3q, | 2q, 4q | 4p, 9q, | 1q, 3q, | 2q, 4q | 4p, 9q, |
| cancer | | 8p | | 16q, 18q | 8q, 9p, | | 11p, 11q, | 8p, 15q | | 11p, 11q, | 8p, 8q, | | 11p, 11q, |
| | | | | | 15q | | 16q, 18q | | | 16q, 18q | 9p, 15q | | 16q, 18q |
| Cervical | 14 | 1q, 3q, | 2q | 4p, 9q, | 1q, 3q, | 2q, 4q | 4p, 9q, | 1q, 3q, | 2q | 4p, 9q, | 1q, 3q, | 2q, 4q | 4p, 9q, |
| cancer | | 15q | | 11p, 13q | 8p, 8q, | | 11p, 11q, | 9p, 15q | | 11p, 13q, | 8p, 8q, | | 11p, 11q, |
| | | | | | 9p | | 18q | | | 16q, 18q | 9p, 15q | | 13q, 16q, |
| | | | | | | | | | | | | | 18q |
| Cervical | 15 | / | / | 11p16q, | / | / | 2q, 4p, | 1q, 3q, | / | 2q, 4p, | 1q, 3q, | / | 2q, 4p, |
| cancer | | | | 18q | | | 4q, 11q, | 8p | | 4q, 9q, | 8p, 8q, | | 4q, 9q, |
| | | | | | | | 15q, 16q, | | | 11p16q, | 9p | | 11p, 11q, |
| | | | | | | | 18q | | | 18q | | | 15q, 16q, |
| | | | | | | | | | | | | | 18q |
| CIN3 | 16 | / | / | / | 3q | / | / | / | / | 3q, 4q, | 3q | / | 4q, 6q, |
| | | | | | | | | | | 6q, 8p,, | | | 8p, 8q, |
| | | | | | | | | | | 11q, 18q | | | 10q, 11q, |
| | | | | | | | | | | | | | 18q |
| CIN3 | 17 | / | / | / | / | / | / | 3q, 5p | / | 4p, 5q, | 3q, 5p | / | 4p, 4q, |
| | | | | | | | | | | 6p, 8q, | | | 5q, 6p, |
| | | | | | | | | | | 10p, 11q, | | | 6q, 8p, |
| | | | | | | | | | | 18q | | | 8q, 10p, |
| | | | | | | | | | | | | | 10q, 11q, |
| | | | | | | | | | | | | | 18q |
| CIN3 | 18 | / | / | / | 3q | / | / | / | / | / | 3q | / | / |
| CIN3 | 19 | / | / | / | / | / | / | / | / | / | / | / | / |
| CIN3 | 20 | / | / | / | / | / | / | 3q | / | / | 3q | / | / |
| CIN2 | 21 | / | / | / | / | / | 6p, 6q | / | / | 6p, 6q | / | / | 6p, 6q |
| CIN2 | 22 | / | / | / | / | / | / | / | / | / | / | / | / |
| CIN2 | 23 | / | / | / | / | / | / | 3q | / | / | 3q, 4q | / | / |
| CIN2 | 24 | / | / | / | / | / | / | / | / | / | / | / | / |
| CIN2 | 25 | / | / | / | / | / | / | / | / | / | 3q | / | / |
| CIN1 | 26 | / | / | / | / | / | / | / | / | / | / | / | / |
| CIN1 | 27 | / | / | / | / | / | / | / | / | / | / | / | / |
| CIN1 | 28 | / | / | / | / | / | / | / | / | / | / | / | / |
| CIN1 | 29 | / | / | / | / | / | / | / | / | / | / | / | / |
| CIN1 | 30 | / | / | / | / | / | / | / | / | / | / | / | / |

Results analysis: in the library of cervical cancer samples, the detection rate of primer 1 was 8/15=53.5%, and the detection rate of cervical cancer in the case where amplicon library construction was performed using primer 2 was 10/15=66.7%. The detection rate of primer 3 was 13/15=86.7%, and the detection rate of cervical cancer in the case where amplicon library construction was performed using primer 4 was 15/15=100%. In CIN3, the detection rate of primer 1 was 0/5=0%, the detection rate of primer 2 was 2/5=40%; the detection rate of primer 3 was 3/5=60%, and the detection rate of primer 4 was 4/5=80%.

In CIN2, the detection rate of primer 1 was 0/5=0%, the detection rate of primer 2 was 1/5=20%; the detection rate in the library constructed with primer 3 was 2/5=40%, and the detection rate of primer 4 was 3/5=60%. No sites were detected in the CIN1 sample using library constructed with four kinds of primers. Primer 4 (with G added at the 5′ end in combination with phosphorylation) could detect more chromosomal abnormalities.

### Example 14 Detection of Ploidy in Urothelial Cancer Samples

20 cases of urothelial cancer samples with clear pathological information were collected, and subjected to different methods for ploidy library construction. The four kinds of primers in Table 18 were used. The PCR enzyme used was KAPA's multiplex enzyme (no need to add A to the product), and NEB library construction kit was used for adaptor ligation and library pre-amplification. The detection results are shown in Table 13.

TABLE 13
Detection of ploidy in urothelial cancer samples

| Sam- | | | | Primer 2: Primers with | | | Primer 3: Primers with G added to | | | Primer 4: Primers with G added to 5′ | | |
|---|---|---|---|---|---|---|---|---|---|---|---|---|
| ple | Primer 1: Ordinary primer | | | phosphorylated 5′ end | | | 5′ end | | | end and phosphorylated | | |
| No. | GAIN | LOSS | AI | GAIN | LOSS | AI | GAIN | LOSS | AI | GAIN | LOSS | AI |
| 1 | 1q | / | 4p, 9q | 1q, 7q, | / | 4p, 9q, | 1q, 7q | / | 4p, 11q, | 1q, 7q, | / | 4p, 9q, |
| | | | | 8q | | 16q, 18p | | | 13q, 16q, | 8q, 17q, | | 11p, 11q, |
| | | | | | | | | | 18p | 18q | | 13q, 16q, |
| | | | | | | | | | | | | 18p |
| 2 | / | / | / | / | 4p | 4p, 4q, | / | 4p | 4p, 4q, | / | 4p | 4p, 4q, |
| | | | | | | 5q, 11q, | | | 5q, 8p, | | | 5q, 6p, |
| | | | | | | 18q | | | 8q, 10p, | | | 6q, 8p, |
| | | | | | | | | | 10q, 11q, | | | 8q, 10p, |
| | | | | | | | | | 18q | | | 10q, 11q, |
| | | | | | | | | | | | | 18q |
| 3 | / | / | / | / | / | 4q, 5q, | / | / | 4p, 4q, | 7q, 8q, | / | 4p, 4q, |
| | | | | | | 6p, 6q, | | | 6p, 6q, | | | 5q, 6p, |
| | | | | | | | | | | | | 6q, |
| 4 | / | 11p, 11q | / | / | / | / | / | 11p, 11q | / | / | 11p, 11q | / |
| 5 | 1q, 17q | 11q, | / | 1q, 17q, | 11q, 13q, | / | 1q, 17q, | 11q | / | 1q, 17q, | 11q, 13q, | / |
| | | | | 18q | | | 18q | | | 18q | | |
| 6 | / | / | 4q, 5q, | / | / | 4q, 5q, | / | / | 4q, 5q, | / | / | 4q, 5q, |
| | | | 6p, 6q | | | 6p | | | 6q | | | 6p, 6q |
| 7 | / | / | / | / | 11q, 13q, | / | 5q | / | / | / | 11q, 13q, | / |
| 8 | 18q | / | / | 18q | / | / | 18q | / | / | 18q | / | / |
| 9 | 17q, | / | 4p, 6p, | 17q, 18q | / | 4p, 6q, | 17q, 18q | / | 4p, 6p, | 17q, 18q | 11p, 11q | 4p, 6p, |
| | 18q | | 6q, 8p, | | | 8p, 18q | | | 6q, 8p, | | | 6q, 8p, |
| | | | 10q | | | | | | 10q, 11q, | | | 10q, 11q, |
| | | | | | | | | | 18q | | | 18q |
| 10 | / | / | / | / | / | / | / | / | / | / | / | / |
| 11 | 17q | 11q, 13q, | 1p, 14q, | 17q | / | 1p, 3q, | 17q | 11q, 13q, | 1p, 3q, | 17q | 11q, 13q, | 1p, 3q, |
| | | | 18p, 18q, | | | 5p, 5q, | | | 5p, 5q, | | | 5p, 5q, |
| | | | 22q | | | 8p, 8q, | | | 8p, 8q, | | | 8p, 8q, |
| | | | | | | 11p, 11q, | | | 11p, 14q, | | | 11p, 11q, |
| | | | | | | 18q, 22q | | | 18q, 22q | | | 14q, 18p, |
| | | | | | | | | | | | | 18q, 22q |
| 12 | / | / | / | / | / | / | / | / | / | / | 13q, | / |
| 13 | | / | 4q, 5q, | | / | / | / | / | 4q, 5q, | | 4p | 4q, 5q, |
| | | | 6p, 6q, | | | | | | 6p, 6q, | | | 6p, 6q, |
| 14 | / | / | / | 18q | / | / | 1q, 17q, | / | / | 18q | / | / |
| | | | | | | | 18q | | | | | |
| 15 | 1q, 18q | / | 1p, 3q, | 1q, 17q, | / | 1p, 3q, | 1q, 18q | / | 1p, 3q, | 1q, 17q, | / | 1p, 3q, |
| | | | 5p, | 18q | | 5p, 11q, | | | 5p, 11q, | 18q | | 5p, 11q, |
| | | | 14q18q, | | | 18p, 18q, | | | 14q18q, | | | 14q, 18p, |
| | | | 22q | | | 22q | | | 22q | | | 18q, 22q |
| 16 | / | / | / | / | / | / | / | / | / | / | / | / |
| 17 | / | / | / | / | / | / | / | / | / | / | 9q, 13q, | / |
| 18 | 7q | / | 18p | 7q, 8q, | / | 16q, 18p | 7q | / | 16q, 18p | 7q, 8q, | / | 16q, 18p |
| 19 | / | 11q | 4p, 6p, | / | 11q, 13q, | 6q, 8p, | / | 11q | 4p, 6p, | / | 11q, 13q, | 4p, 6p, |
| | | | 6q, 8p, | | | 10q, 11q, | | | 6q, 8p, | | | 6q, 8p, |
| | | | | | | 18q | | | 10q, | | | 10q, 11q, |
| | | | | | | | | | | | | 18q |
| 20 | / | / | / | 17q | / | 16q | / | / | 16q, 18q | 17q | / | 16q, 18q |
| 21 | / | / | / | / | 8p, 11q | 4p, 6p, | / | 8p, 11q | 4p, 8p, | / | 8p, 11q | 4p, 6p, |
| | | | | | | 6q, 11q, | | | 10q, 18q | | | 6q, 8p, |
| | | | | | | 18q | | | | | | 10q, 11q, |
| | | | | | | | | | | | | 18q |
| 22 | 17q, | / | 8p, 11q, | 17q, 18q | / | 3q, 5p, | 17q, 18q | / | 3q, 5p, | 17q, 18q | / | 3p, 5p, |
| | 18q | | 14q, 18p, | | | 8p, 11q, | | | 11q, 14q, | | | 8p, 8q, |
| | | | 18q | | | 14q, 18p, | | | 18p, 18q | | | 11p, 11q, |
| | | | | | | 18q | | | | | | 14q, 18p, |
| | | | | | | | | | | | | 18q |
| 23 | / | / | / | / | / | / | / | / | / | 7q, | / | / |
| 24 | / | 9q, 13q, | 8q, | / | 9q, 13q, | 8q, 11p | / | 9q, 13q, | 8q, 11p | / | 9q, 13q, | 8q, 11p |
| 25 | 1q, 17q, | / | 5q, 8p, | 1q, 17q, | / | 1p, 5p, | 1q, 17q, | / | 5q, 8p, | 1q, 17q, | / | 1p, 5p, |
| | 18q | | 8q, 11p | 18q | | 5q, 11p, | 18q | | 11p, 11q, | 18q | | 5q, 8p, |
| | | | | | | 11q, 18q, | | | 18q, 22q | | | 8q, 11p, |
| | | | | | | 22q | | | | | | 11q, 18q, |
| | | | | | | | | | | | | 22q |

Results analysis: in the library of urothelial cancer samples, the detection rate of chromosome ploidy changes in the case where amplicon library construction was performed using primer 1 was 14/25=56%; the detection rate of chromosome ploidy changes in the case where amplicon library construction was performed using primer 2 was 18/25=72%, the detection rate of chromosome ploidy changes in the case where amplicon library construction was performed using primer 3 was 21/25=84%; the detection rate of chromosome ploidy changes in the case where amplicon library construction was performed using primer 4 was 23/25=92%. The amplicon library construction using primer 4 (with G added at the 5′ end in combination with phosphorylation) could lead more chromosomal abnormalities being detected.

### Example 15: RNA Fusion Detection

RNA fusion reference: Seraseq® Fusion RNA Mix v4 were blended with wild-type (fusion-negative) cell line GM 12878 RNA with confirmed genetic background at a concentration of 25 ng/μL in different volumes, and the lower detection limits of library construction method with four different modified primers in Table 19 were investigated. RNA was reverse transcribed using SuperScript™ VILO™ cDNA synthesis kit, and QIAGEN kit was used for cDNA purification. QIAGEN multiplex enzymes were used in multiplex PCR (no need to add A to the product), and Vazyme library construction kit was used for adaptor ligation and library pre-amplification. Each fusion type was tested 10 times at different blending ratios. The detection results are shown in Table 14.

TABLE 14
Detection results of 4 kinds of primers

| | | Primer 1: | Primer 2: modified | | Primer 4: modified |
|---|---|---|---|---|---|
| Blending | | Ordinary | with phosphorylated | Primer 3: with G | with G added to 5′ end |
| concentration | Fusion type | primer | 5′ end | added to 5′ end | and phosphorylated |
| 10% | EML4-ALK | 30/30 | 30/30 | 30/30 | 30/30 |
| | CD74-ROS1 | 28/30 | 30/30 | 30/30 | 30/30 |
| | CCDC6-RET | 29/30 | 30/30 | 30/30 | 30/30 |
| | NCOA4-RET | 26/30 | 27/30 | 28/30 | 30/30 |
| | TPM3-NTRK1 | 28/30 | 29/30 | 30/30 | 30/30 |
| 5% | EML4-ALK | 26/30 | 27/30 | 28/30 | 30/30 |
| | CD74-ROS1 | 28/30 | 30/30 | 30/30 | 30/30 |
| | CCDC6-RET | 27/30 | 27/30 | 29/30 | 30/30 |
| | NCOA4-RET | 26/30 | 27/30 | 28/30 | 30/30 |
| | TPM3-NTRK1 | 24/30 | 26/30 | 28/30 | 30/30 |
| 2.5% | EML4-ALK | 22/30 | 24/30 | 26/30 | 30/30 |
| | CD74-ROS1 | 20/30 | 21/30 | 26/30 | 30/30 |
| | CCDC6-RET | 19/30 | 21/30 | 23/30 | 25/30 |
| | NCOA4-RET | 15/30 | 17/30 | 22/30 | 27/30 |
| | TPM3-NTRK1 | 16/30 | 18/30 | 21/30 | 26/30 |
| 1.25% | EML4-ALK | 15/30 | 20/30 | 24/30 | 26/30 |
| | CD74-ROS1 | 16/30 | 21/30 | 26/30 | 29/30 |
| | CCDC6-RET | 13/30 | 14/30 | 17/30 | 21/30 |
| | NCOA4-RET | 10/30 | 12/30 | 16/30 | 22/30 |
| | TPM3-NTRK1 | 10/30 | 12/30 | 18/30 | 24/30 |
| 0% | EML4-ALK | 0/30 | 0/30 | 0/30 | 0/30 |
| | CD74-ROS1 | 0/30 | 0/30 | 0/30 | 0/30 |
| | CCDC6-RET | 0/30 | 0/30 | 0/30 | 0/30 |
| | NCOA4-RET | 0/30 | 0/30 | 0/30 | 0/30 |
| | TPM3-NTRK1 | 0/30 | 0/30 | 0/30 | 0/30 |

Result analysis: Detection for five fusion types was performed. When the blending concentration was 0%, it was substantially undetectable for both methods. When the blending concentration was 10%, the cumulative detection rate of primer 1 was 94%. The cumulative detection rate of primer 2 was 97.3%, the cumulative detection rate of primer 3 was 98.7%, and the cumulative detection rate of primer 4 was 100%; in the case of 5% blending concentration, the cumulative detection rate of primer 1 was 87.3%, and the cumulative detection rate of primer 2 was 91.3%, the cumulative detection rate of primer 3 was 95.3%, and the cumulative detection rate of primer 4 was 100%6; in the case of 2.5% blending concentration, the cumulative detection rate of primer 1 was 61.3%, the cumulative detection rate of primer 2 was 67.3%, and the cumulative detection rate of primer 3 was 78.7%, and the cumulative detection rate of primer 4 was 92%; in the case of 1.25% blending concentration, the cumulative detection rate of primer 1 was 42.7%, the detection rate of primer 2 was 52.6%, the cumulative detection rate of primer 3 was 67.3%, and the cumulative detection rate of primer 4 was 81.3%. It could be seen from the above results that the effect of primer 4 was better than primer 1 when detecting low-frequency fusion.

### Example 16 Detection of Pathogenic Microorganisms

Amplicon regions to be detected were integrated into the same plasmid containing the T7 promoter. After performing in vitro transcription, an RNA reference was obtained. The size and concentration of the target fragment were detected by 2100, and then converted into a concentration in copy number. References with different copy numbers were used to evaluate the detection capabilities of amplicons for different modified primers. Reverse transcription was performed using SuperScript IV reverse transcriptase, and QIAGEN kit was used for cDNA purification. Multiplex PCR was performed by four kinds of primers in Table 20, respectively, KAPA multiplex enzyme was used in multiplex PCR (no need to add A to the product), and NEB library construction kit was used for adaptor ligation and library pre-amplification. Detection was performed 10 times for different copy numbers. The detection results are shown in Table 15.

TABLE 15
Detection of 4 different primers

| Initial copy number | | | | | |
|---|---|---|---|---|---|
| of reverse | | Primer 1: | Primer 2: modified | | Primer 4: modified with |
| transcriptional | Pathogenic | Ordinary | with phosphorylated | Primer 3: with G | G added to 5′ end and |
| reaction | microorganism type | primer | 5′ end | added to 5′ end | phosphorylated |
| 10 | Influenza A virus | 10/10 | 10/10 | 10/10 | 10/10 |
| | influenza B virus | 10/10 | 10/10 | 10/10 | 10/10 |
| | covid-19 | 10/10 | 10/10 | 10/10 | 10/10 |
| 5 | Influenza A virus | 10/10 | 10/10 | 10/10 | 10/10 |
| | influenza B virus | 9/10 | 9/10 | 9/10 | 10/10 |
| | covid-19 | 9/10 | 10/10 | 10/10 | 10/10 |
| 2.5 | Influenza A virus | 7/10 | 8/10 | 9/10 | 10/10 |
| | influenza B virus | 6/10 | 7/10 | 8/10 | 9/10 |
| | covid-19 | 7/10 | 8/10 | 9/10 | 10/10 |
| 0 | Influenza A virus | 0/10 | 0/10 | 0/10 | 0/10 |
| | influenza B virus | 0/10 | 0/10 | 0/10 | 0/10 |
| | covid-19 | 0/10 | 0/10 | 0/10 | 0/10 |

Result analysis: the detection capabilities of three pathogenic microorganisms at different copy numbers (reverse transcription reaction) were examined: for 10 copies, it could be detected in all 10 tests; for 5 copies, the detection rate of primer 1 was 93.3%, the detection rate of primer 2 was 96.7n, the detection rate of primer 3 was 96.7%, the detection rate of primer 4 was 100% for 2.5 copies, the detection rate of primer 1 was 66.7%, the detection rate of primer 2 was 76.7%, the detection rate of primer 3 was 86.7%, and the detection rate of primer 4 was 96.7%; for 0 copies, two primers were tested negative. Detection of low-concentration pathogenic microorganisms with primer 4 showed a better effect than primer 1.

### Example 17: Comparison of TA Cloning Efficiency of PCR Products with Different Primers

Primers synthesized in different ways as shown in Table 21 were used, to amplify the promoter region of the purg gene of zebrafish. Three TA clones were tested in parallel for each primer type. PCR products were subjected to TA cloning according to the method in Example 1. The number of white spots (confirmation of insertion) and cloning efficiency (white spot rate) are detailed in Table 16 below.

TABLE 16
Cloning efficiency of PCR products with different primers

| | Parallel 1 | | Parallel 2 | | Parallel 3 | | Total | |
|---|---|---|---|---|---|---|---|---|
| | Number | | Number | | Number | | Number | |
| Primer type used for | of white | Cloning | of white | Cloning | of white | cloning | of white | Cloning |
| amplification | spots | efficiency | spots | efficiency | spots | efficiency | spots | efficiency |
| Ordinary primer | 415 | 46% | 408 | 44% | 426 | 47% | 1249 | 46% |
| Primers with | 505 | 58% | 512 | 56% | 509 | 59% | 1526 | 57.7% |
| phosphorylated 5′ end | | | | | | | | |
| Primers with G added to | 613 | 71% | 603 | 70% | 626 | 72% | 1842 | 71% |
| 5′ end | | | | | | | | |
| Primers with G added to | 723 | 81% | 736 | 85% | 729 | 82% | 2188 | 82.7% |
| 5′ end and phosphorylated | | | | | | | | |

Result analysis: ordinary primers had the worst effect on the number of white spots and cloning efficiency, and the primers with G added to 5′ end and phosphorylated had the best effect. Because all other control conditions in the experiment (transfection, plating, culture conditions, etc.) were the same, and the single variable was different primers, it showed that products with primers with added to 5′ end and phosphorylated have higher TA ligation efficiency.

The primers used in each example were specifically as follows.

TABLE 17
Mutation detection primers

| | | | | | | | 2: Primers with | | 3: Primers with | | 4: Primers with | |
|---|---|---|---|---|---|---|---|---|---|---|---|---|
| | | | | | | | phosphorylated | | G added | | G added to 5′ end | |
| | | | | | 1: Ordinary primer | | 5′ end | | to 5′ end | | and phosphorylated | |
| | | | | | Fwd_ | Rev_ | Fwd_ | Rev_ | Fwd_ | Rev_ | Fwd_ | Rev_ |
| No | Gene | Chr | Amplicon_Start | Amplicon_Stop | Primer | Primer | Primer | Primer | Primer | Primer | Primer | Primer |
| 1 | CTNNB1 | chr3 | 41266108 | 41266224 | CAGTCTT | CTCTTAC | CAGTCTT | CTCTTACC | GCAGTCT | GCTCTTAC | GCAGTCTT | GCTCTTA |
| | | | | | ACCTGGA | CAGCTAC | ACCTGGA | AGCTACTT | TACCTGG | CAGCTACT | ACCTGGAC | CCAGCTA |
| | | | | | CTCTGGA | TTGTTCTT | CTCTGGA | GTTCTTGA | ACTCTGG | TGTTCTTG | TCTGGAAT | CTTGTTC |
| | | | | | ATC | GAGTG | ATC | GTG | AATC | AGTG | C | TTGAGTG |
| 2 | DNAH2 | chr17 | 7690208 | 7690307 | GTTTCCTC | GCCGTTG | GTTTCCTC | GCCGTTGA | GTTTCCT | GCCGTTGA | GTTTCCTC | GCCGTTG |
| | | | | | ATCACCC | ATGAGGG | ATCACCC | TGAGGGT | CATCACC | TGAGGGTC | ATCACCCA | ATGAGG |
| | | | | | ACCATCT | TCAACA | ACCATCT | CAACA | CACCATC | AACA | CCATCT | GTCAACA |
| | | | | | | | | | T | | | |
| 3 | ERBB3 | chr12 | 56478889 | 56479009 | CCAGGTC | CATCATC | CCAGGTC | CATCATCA | GCCAGGT | GCATCATC | GCCAGGT | GCATCAT |
| | | | | | TACGATG | AAGGAGG | TACGATG | AGGAGGT | CTACGAT | AAGGAGGT | CTACGATG | CAAGGA |
| | | | | | GGAAGTT | TACCAGT | GGAAGTT | ACCAGTCT | GGGAAGT | ACCAGTCT | GGAAGTTT | GGTACCA |
| | | | | | T | CTTG | T | TG | TT | TG | | GTCTTG |
| 4 | FBXW7 | chr4 | 153249284 | 153249398 | ATTTAAG | GAGAATG | ATTTAAG | GAGAATG | GATTTAA | GAGAATGT | GATTTAAG | GAGAAT |
| | | | | | AGCACAC | TATACAC | AGCACAC | TATACACA | GAGCACA | ATACACAC | AGCACACT | GTATACA |
| | | | | | TGTCACT | ACCTTAT | TGTCACT | CCTTATAT | CTGTCAC | CTTATATG | GTCACTAT | CACCTTA |
| | | | | | ATTTCAGT | ATGGGCA | ATTTCAG | GGGCA | TATTTCA | GGCA | TTCAGT | TATGGGC |
| | | | | | | | T | | GT | | | A |
| 5 | PIK3CA | chr3 | 178936019 | 178936103 | ATTTTACA | GCACTTA | ATTTTAC | GCACTTAC | GATTTTA | GCACTTAC | GATTTTAC | GCACTTA |
| | | | | | GAGTAAC | CCTGTGA | AGAGTAA | CTGTGACT | CAGAGTA | CTGTGACT | AGAGTAA | CCTGTGA |
| | | | | | AGACTAG | CTCCATA | CAGACTA | CCATAGA | ACAGACT | CCATAGAA | CAGACTA | CTCCATA |
| | | | | | CTAGAGA | GAAAAT | GCTAGAG | AAAT | AGCTAGA | AAT | GCTAGAG | GAAAAT |
| | | | | | CA | | ACA | | GACA | | ACA | |
| 6 | PIK3CA | chr3 | 178936019 | 178936103 | ATTTTACA | GCACTTA | ATTTTAC | GCACTTAC | GATTTTA | GCACTTAC | GATTTTAC | GCACTTA |
| | | | | | GAGTAAC | CCTGTGA | AGAGTAA | CTGTGACT | CAGAGTA | CTGTGACT | AGAGTAA | CCTGTGA |
| | | | | | AGACTAG | CTCCATA | CAGACTA | CCATAGA | ACAGACT | CCATAGAA | CAGACTA | CTCCATA |
| | | | | | CTAGAGA | GAAAAT | GCTAGAG | AAAT | AGCTAGA | AAT | GCTAGAG | GAAAAT |
| | | | | | CA | | ACA | | GACA | | ACA | |
| 7 | PIK3CA | chr3 | 178936092 | 178936169 | CGAGATC | GCTGAGA | CGAGATC | GCTGAGA | GCGAGAT | GCTGAGAT | GCGAGAT | GCTGAGA |
| | | | | | CTCTCTCT | TCAGCCA | CTCTCTCT | TCAGCCA | CCTCTCT | CAGCCAAA | CCTCTCTC | TCAGCCA |
| | | | | | GAAATCA | AATTCAG | GAAATCA | AATTCAGT | CTGAAAT | TTCAGTTA | TGAAATCA | AATTCAG |
| | | | | | CTGA | TTATTTTT | CTGA | TATTTTT | CACTGA | TTTTT | CTGA | TTATTTT |
| | | | | | | | | | | | | T |
| 8 | PIK3CA | chr3 | 178916807 | 178916900 | CCCTCCAT | CCTACTG | CCCTCCA | CCTACTGG | GCCCTCC | GCCTACTG | GCCCTCCA | GCCTACT |
| | | | | | CAACTTCT | GTTCAAT | TCAACTT | TTCAATTA | ATCAACT | GTTCAATT | TCAACTTC | GGTTCAA |
| | | | | | TCAAGAT | TACTTTT | CTTCAAG | CTTTTAAA | TCTTCAA | ACTTTTAA | TTCAAGAT | TTACTTT |
| | | | | | GA | AAAAAGG | ATGA | AAGGGT | GATGA | AAAGGGT | GA | TAAAAA |
| | | | | | | GT | | | | | | GGGT |
| 9 | PIK3CA | chr3 | 178952062 | 178952185 | TGAGCAA | AGAGTTA | TGAGCAA | AGAGTTAT | GTGAGCA | GAGAGTTA | GTGAGCA | GAGAGTT |
| | | | | | GAGGCTT | TTAACAG | GAGGCTT | TAACAGT | AGAGGCT | TTAACAGT | AGAGGCTT | ATTAACA |
| | | | | | TGGAGTA | TGCAGTG | TGGAGTA | GCAGTGT | TTGGAGT | GCAGTGTG | TGGAGTAT | GTGCAGT |
| | | | | | TTTC | TGGAAT | TTTC | GGAAT | ATTTC | GAAT | TTC | GTGGAAT |
| 10 | PIK3CA | chr3 | 178917406 | 178917512 | GTGATCTT | AGTCCTG | GTGATCT | AGTCCTGT | GTGATCT | GAGTCCTG | GTGATCTT | GAGTCCT |
| | | | | | CCAAATC | TACTTCT | TCCAAAT | ACTTCTGG | TCCAAAT | TACTTCTG | CCAAATCT | GTACTTC |
| | | | | | TACAGAG | GGATCTT | CTACAGA | ATCTTTAA | CTACAGA | GATCTTTA | ACAGAGTT | TGGATCT |
| | | | | | TTCCC | TAACCAT | GTTCCC | CCAT | GTTCCC | ACCAT | CCC | TTAACCA |
| | | | | | | | | | | | | T |
| 11 | PPP2R1A | chr19 | 52715903 | 52716027 | CTTGCTCC | GATCTCA | CTTGCTCC | GATCTCAC | GCTTGCT | GATCTCAC | GCTTGCTC | GATCTCA |
| | | | | | TCTCTGCC | CTCTTGA | TCTCTGCC | TCTTGACG | CCTCTCT | TCTTGACG | CTCTCTGC | CTCTTGA |
| | | | | | ATACTG | CGTTGTC | ATACTG | TTGTCCA | GCCATAC | TTGTCCA | CATACTG | CGTTGTC |
| | | | | | | CA | | | TG | | | CA |
| 12 | PTEN | chr10 | 89692795 | 89692910 | GTTGCAC | CCCGATG | GTTGCAC | CCCGATGT | GTTGCAC | GCCCGATG | GTTGCACA | GCCCGAT |
| | | | | | AATATCC | TAATAAA | AATATCC | AATAAAT | AATATCC | TAATAAAT | ATATCCTT | GTAATAA |
| | | | | | TTTTGAA | TATGCAC | TTTTGAA | ATGCACAT | TTTTGAA | ATGCACAT | TTGAAGAC | ATATGCA |
| | | | | | GACCAT | ATATCAT | GACCAT | ATCATTAC | GACCAT | ATCATTAC | CAT | CATATCA |
| | | | | | | TACAC | | AC | | AC | | TTACAC |
| 13 | PTEN | chr10 | 89717567 | 89717693 | ATCGTTTT | CGGCTGA | ATCGTTTT | CGGCTGA | GATCGTT | GCGGCTGA | GATCGTTT | GCGGCTG |
| | | | | | TGACAGT | GGGAACT | TGACAGT | GGGAACT | TTTGACA | GGGAACTC | TTGACAGT | AGGGAA |
| | | | | | TTGACAG | CAAAGT | TTGACAG | CAAAGT | GTTTGAC | AAAGT | TTGACAGT | CTCAAAG |
| | | | | | TTAAAGG | | TTAAAGG | | AGTTAAA | | TAAAGG | T |
| | | | | | | | | | GG | | | |
| 14 | SMO | chr7 | 128846335 | 128846451 | GGACTCT | CCCAGTA | GGACTCT | CCCAGTAT | GGACTCT | GCCCAGTA | GGACTCTG | GCCCAGT |
| | | | | | GTGAGTG | TATTTTG | GTGAGTG | ATTTTGTT | GTGAGTG | TATTTTGTT | TGAGTGG | ATATTTT |
| | | | | | GGATTTG | TTGCCCA | GGATTTG | GCCCAACT | GGATTTG | GCCCAACT | GATTTGT | GTTGCCC |
| | | | | | T | ACTG | T | G | T | G | | AACTG |
| 15 | TP53 | chr17 | 7578241 | 7578366 | AAAGTGT | CTGCTCA | AAAGTGT | CTGCTCAG | GAAAGTG | GCTGCTCA | GAAAGTG | GCTGCTC |
| | | | | | TTCTGTCA | GATAGCG | TTCTGTCA | ATAGCGA | TTTCTGTC | GATAGCGA | TTTCTGTC | AGATAGC |
| | | | | | TCCAAAT | ATGGTGA | TCCAAAT | TGGTGA | ATCCAAA | TGGTGA | ATCCAAAT | GATGGTG |
| | | | | | ACTCCA | | ACTCCA | | TACTCCA | | ACTCCA | A |
| 16 | TP53 | chr17 | 7577508 | 7577617 | GGCTCCT | CCTCATC | GGCTCCT | CCTCATCT | GGCTCCT | GCCTCATC | GGCTCCTG | GCCTCAT |
| | | | | | GACCTGG | TTGGGCC | GACCTGG | TGGGCCTG | GACCTGG | TTGGGCCT | ACCTGGA | CTTGGGC |
| | | | | | AGTCTT | TGTGTT | AGTCTT | TGTT | AGTCTT | GTGTT | GTCTT | CTGTGTT |
| 17 | TP53 | chr17 | 7577031 | 7577157 | CTTGCTTA | CTTGCTT | CTTGCTTA | CTTGCTTC | GCTTGCT | GCTTGCTT | GCTTGCTT | GCTTGCT |
| | | | | | CCTCGCTT | CTCTTTTC | CCTCGCTT | TCTTTTCC | TACCTCG | CTCTTTTCC | ACCTCGCT | TCTCTTT |
| | | | | | AGTGCT | CTATCCT | AGTGCT | TATCCTGA | CTTAGTG | TATCCTGA | TAGTGCT | TCCTATC |
| | | | | | | GAGT | | GT | CT | GT | | CTGAGT |
| 18 | TP53 | chr17 | 7577031 | 7577157 | CTTGCTTA | CTTGCTT | CTTGCTTA | CTTGCTTC | GCTTGCT | GCTTGCTT | GCTTGCTT | GCTTGCT |
| | | | | | CCTCGCTT | CTCTTTTC | CCTCGCTT | TCTTTTCC | TACCTCG | CTCTTTTCC | ACCTCGCT | TCTCTTT |
| | | | | | AGTGCT | CTATCCT | AGTGCT | TATCCTGA | CTTAGTG | TATCCTGA | TAGTGCT | TCCTATC |
| | | | | | | GAGT | | GT | CT | GT | | CTGAGT |
| 19 | TP53 | chr17 | 7577031 | 7577157 | CTTGCTTA | CTTGCTT | CTTGCTTA | CTTGCTTC | GCTTGCT | GCTTGCTT | GCTTGCTT | GCTTGCT |
| | | | | | CCTCGCTT | CTCTTTTC | CCTCGCTT | TCTTTTCC | TACCTCG | CTCTTTTCC | ACCTCGCT | TCTCTTT |
| | | | | | AGTGCT | CTATCCT | AGTGCT | TATCCTGA | CTTAGTG | TATCCTGA | TAGTGCT | TCCTATC |
| | | | | | | GAGT | | GT | CT | GT | | CTGAGT |
| 20 | KRAS | chr12 | 25398119 | 25398270 | TAAGTAC | AGTGCCT | TAAGTAC | AGTGCCTT | GTAAGTA | GAGTGCCT | GTAAGTA | GAGTGCC |
| | | | | | TCATGAA | TGACGAT | TCATGAA | GACGATA | CTCATGA | TGACGATA | CTCATGAA | TTGACGA |
| | | | | | AATGGTC | ACAGCTA | AATGGTC | CAGCTAAT | AAATGGT | CAGCTAAT | AATGGTCA | TACAGCT |
| | | | | | AGAGAAA | ATTC | AGAGAAA | TC | CAGAGAA | TC | GAGAAAC | AATTC |
| | | | | | C | | C | | AC | | | |
| 21 | NRAS | chr1 | 115258614 | 115258835 | AGATGAT | GGCTCGC | AGATGAT | GGCTCGCC | GAGATGA | GGCTCGCC | GAGATGA | GGCTCGC |
| | | | | | CCGACAA | CAATTAA | CCGACAA | AATTAACC | TCCGACA | AATTAACC | TCCGACAA | CAATTAA |
| | | | | | GTGAGAG | CCCTGA | GTGAGAG | CTGA | AGTGAGA | CTGA | GTGAGAG | CCCTGA |
| | | | | | ACA | | ACA | | GACA | | ACA | |
| 22 | ASXL1 | chr20 | 31025073 | 31025256 | GGTGCGT | AGAGTGC | GGTGCGT | AGAGTGC | GGTGCGT | GAGAGTGC | GGTGCGTT | GAGAGT |
| | | | | | TCTGTCAC | TCCTGCC | TCTGTCA | TCCTGCCT | TCTGTCA | TCCTGCCT | CTGTCACG | GCTCCTG |
| | | | | | GATGA | TAAAGAG | CGATGA | AAAGAGT | CGATGA | AAAGAGT | ATGA | CCTAAAG |
| | | | | | | T | | | | | | AGT |
| 23 | DNMT3A | chr2 | 25468850 | 25469065 | CACACTA | CCCAAGG | CACACTA | CCCAAGG | GCACACT | GCCCAAGG | GCACACT | GCCCAA |
| | | | | | GGAGTGC | TCAAGGA | GGAGTGC | TCAAGGA | AGGAGTG | TCAAGGAG | AGGAGTG | GGTCAAG |
| | | | | | CAGAGTT | GATTATT | CAGAGTT | GATTATTG | CCAGAGT | ATTATTGA | CCAGAGTT | GAGATTA |
| | | | | | | GATGAG | | ATGAG | T | TGAG | | TTGATGA |
| | | | | | | | | | | | | G |
| 24 | IDH2 | chr15 | 90631714 | 90631905 | CACAAAG | GAGCCCA | CACAAAG | GAGCCCA | GCACAAA | GAGCCCAT | GCACAAA | GAGCCCA |
| | | | | | TCTGTGG | TCATCTG | TCTGTGG | TCATCTGC | GTCTGTG | CATCTGCA | GTCTGTGG | TCATCTG |
| | | | | | CCTTGTAC | CAAAAAC | CCTTGTA | AAAAACA | GCCTTGT | AAAACATC | CCTTGTAC | CAAAAA |
| | | | | | T | ATC | CT | TC | ACT | | T | CATC |
| 25 | DNMT3A | chr2 | 25469546 | 25469691 | CAATCAT | TTCCAGC | CAATCAT | TTCCAGCC | GCAATCA | GTTCCAGC | GCAATCAT | GTTCCAG |
| | | | | | GGGCTTG | CTGTCCT | GGGCTTG | TGTCCTGA | TGGGCTT | CTGTCCTG | GGGCTTGT | CCTGTCC |
| | | | | | TTCTGCAC | GACAAC | TTCTGCA | CAAC | GTTCTGC | ACAAC | TCTGCAC | TGACAAC |
| | | | | | | | C | | AC | | | |
| 26 | TET2 | chr4 | 106158000 | 106158210 | ACCCCAA | GGCGTGA | ACCCCAA | GGCGTGA | GACCCCA | GGCGTGAA | GACCCCA | GGCGTGA |
| | | | | | ACTGAGT | AACTGCT | ACTGAGT | AACTGCTT | AACTGAG | ACTGCTTC | AACTGAGT | AACTGCT |
| | | | | | CTTGCCAT | TCAGATG | CTTGCCA | CAGATG | TCTTGCC | AGATG | CTTGCCAT | TCAGATG |
| | | | | | A | | TA | | ATA | | A | |
| 27 | PTPN11 | chr12 | 112926732 | 112926865 | TTAGCATT | CTGGATG | TTAGCAT | CTGGATG | GTTAGCA | GCTGGATG | GTTAGCAT | GCTGGAT |
| | | | | | GTCTCTG | GTTTTGG | TGTCTCTG | GTTTTGGG | TTGTCTCT | GTTTTGGG | TGTCTCTG | GGTTTTG |
| | | | | | AGTCCAC | GAACGTC | AGTCCAC | AACGTCA | GAGTCCA | AACGTCAA | AGTCCACT | GGAACGT |
| | | | | | TAAAA | AATA | TAAAA | ATA | CTAAAA | TA | AAAA | CAATA |
| 28 | RUNX1 | chr21 | 36171738 | 36171891 | GATGGTT | ATTAAAC | GATGGTT | ATTAAACC | GATGGTT | GATTAAAC | GATGGTTG | GATTAAA |
| | | | | | GGATCTG | CCTGGTA | GGATCTG | CTGGTACA | GGATCTG | CCTGGTAC | GATCTGCC | CCCTGGT |
| | | | | | CCTTGTAT | CATAGGC | CCTTGTAT | TAGGCCA | CCTTGTA | ATAGGCCA | TTGTATCT | ACATAGG |
| | | | | | CTG | CACATA | CTG | CATA | TCTG | CATA | G | CCACATA |
| 29 | EGFR | chr7 | 55249000 | 55249131 | CTCCAGG | CAGTTGA | CTCCAGG | CAGTTGA | GCTCCAG | GCAGTTGA | GCTCCAG | GCAGTTG |
| | | | | | AAGCCTA | GCAGGTA | AAGCCTA | GCAGGTA | GAAGCCT | GCAGGTAC | GAAGCCT | AGCAGGT |
| | | | | | CGTGATG | CTGGGAG | CGTGATG | CTGGGAG | ACGTGAT | TGGGAG | ACGTGATG | ACTGGGA |
| | | | | | | | | | G | | | G |
| 30 | KRAS | chr12 | 25398274 | 25398385 | GCTGTAT | AGGTACT | GCTGTAT | AGGTACT | GCTGTAT | GAGGTACT | GCTGTATC | GAGGTA |
| | | | | | CGTCAAG | GGTGGAG | CGTCAAG | GGTGGAG | CGTCAAG | GGTGGAGT | GTCAAGG | CTGGTGG |
| | | | | | GCACTCTT | TATTTGA | GCACTCT | TATTTGAT | GCACTCT | ATTTGATA | CACTCTTG | AGTATTT |
| | | | | | G | TAGTGTA | TG | AGTGTATT | TG | GTGTATT | | GATAGTG |
| | | | | | | TT | | | | | | TATT |
| 31 | CDKN2A | chr9 | 21968163 | 21968300 | CTGTAGG | TGTGCCA | CTGTAGG | TGTGCCAC | GCTGTAG | GTGTGCCA | GCTGTAG | GTGTGCC |
| | | | | | ACCTTCG | CACATCT | ACCTTCG | ACATCTTT | GACCTTC | CACATCTT | GACCTTCG | ACACATC |
| | | | | | GTGACTG | TTGACC | GTGACTG | GACC | GGTGACT | TGACC | GTGACTG | TTTGACC |
| | | | | | | | | | G | | | |
| 32 | ERBB2 | chr17 | 37868148 | 37868263 | TGTTCCAT | GGGTATG | TGTTCCAT | GGGTATGT | GTGTTCC | GGGTATGT | GTGTTCCA | GGGTATG |
| | | | | | CCTCTGCT | TGGCTAC | CCTCTGCT | GGCTACAT | ATCCTCT | GGCTACAT | TCCTCTGC | TGGCTAC |
| | | | | | GTCA | ATGTTCC | GTCA | GTTCCT | GCTGTCA | GTTCCT | TGTCA | ATGTTCC |
| | | | | | | T | | | | | | T |
| 33 | FGFR3 | chr4 | 1803545 | 1803665 | GTCATCT | TGCGTCA | GTCATCT | TGCGTCAC | GTCATCT | GTGCGTCA | GTCATCTG | GTGCGTC |
| | | | | | GCCCCCA | CTGTACA | GCCCCCA | TGTACACC | GCCCCCA | CTGTACAC | CCCCCACA | ACTGTAC |
| | | | | | CAGA | CCTTGC | CAGA | TTGC | CAGA | CTTGC | GA | ACCTTGC |
| 34 | HRAS | chr11 | 533804 | 533926 | GTGGTCA | GATGGCA | GTGGTCA | GATGGCA | GTGGTCA | GATGGCAA | GTGGTCAT | GATGGCA |
| | | | | | TTGATGG | AACACAC | TTGATGG | AACACAC | TTGATGG | ACACACAC | TGATGGG | AACACAC |
| | | | | | GGAGAC | ACAGGA | GGAGAC | ACAGGA | GGAGAC | AGGA | GAGAC | ACAGGA |
| 35 | KRAS | chr12 | 25378532 | 25378655 | TTTCAGTG | GGAAATA | TTTCAGT | GGAAATA | GTTTCAG | GGAAATAA | GTTTCAGT | GGAAAT |
| | | | | | TTACTTAC | AATGTGA | GTTACTT | AATGTGAT | TGTTACT | ATGTGATT | GTTACTTA | AAATGTG |
| | | | | | CTGTCTTG | TTTGCCT | ACCTGTC | TTGCCTTC | TACCTGT | TGCCTTCT | CCTGTCTT | ATTTGCC |
| | | | | | TCTT | TCT | TTGTCTT | T | CTTGTCTT | | GTCTT | TTCT |
| 36 | MET | chr7 | 116423378 | 116423501 | TTTTGAGT | TCAAGGT | TTTTGAGT | TCAAGGTT | GTTTTGA | GTCAAGGT | GTTTTGAG | GTCAAG |
| | | | | | TTGCAGA | TGCTGAT | TTGCAGA | GCTGATTT | GTTTGCA | TGCTGATT | TTTGCAGA | GTTGCTG |
| | | | | | CTTTCCA | TTTGGTC | CTTTCCA | TGGTC | GACTTTC | TTGGTC | CTTTCCA | ATTTTGG |
| | | | | | | | | | CA | | | TC |
| 37 | MLL | chr11 | 118359341 | 118359461 | GTTGCCTT | CAAGTCT | GTTGCCTT | CAAGTCTG | GTTGCCT | GCAAGTCT | GTTGCCTT | GCAAGTC |
| | | | | | CCACAAA | GTTGTGA | CCACAAA | TTGTGAGC | TCCACAA | GTTGTGAG | CCACAAA | TGTTGTG |
| | | | | | CGTG | GCCCTTC | CGTG | CCTTC | ACGTG | CCCTTC | CGTG | AGCCCTT |
| | | | | | | | | | | | | C |
| 38 | PIK3CA | chr3 | 178916781 | 178916899 | GAAAAAG | CCCCCTC | GAAAAAG | CCCCCTCC | GAAAAAG | GCCCCCTC | GAAAAAG | GCCCCCT |
| | | | | | CCGAAGG | CATCAAC | CCGAAGG | ATCAACTT | CCGAAGG | CATCAACT | CCGAAGG | CCATCAA |
| | | | | | TCACAA | TTCTTC | TCACAA | CTTC | TCACAA | TCTTC | TCACAA | CTTCTTC |
| 39 | TP53 | chr17 | 7572893 | 7573009 | GAGGCTG | GCCACCT | GAGGCTG | GCCACCTG | GAGGCTG | GCCACCTG | GAGGCTGT | GCCACCT |
| | | | | | TCAGTGG | GAAGTCC | TCAGTGG | AAGTCCA | TCAGTGG | AAGTCCAA | CAGTGGG | GAAGTCC |
| | | | | | GGAAC | AAAAAG | GGAAC | AAAAG | GGAAC | AAAG | GAAC | AAAAAG |
| 40 | VHL | chr3 | 10183506 | 10183620 | CCGCCGT | CCCGGGT | CCGCCGT | CCCGGGT | GCCGCCG | GCCCGGGT | GCCGCCGT | GCCCGG |
| | | | | | CTTCTTCA | GGTCTGG | CTTCTTCA | GGTCTGG | TCTTCTTC | GGTCTGGA | CTTCTTCA | GTGGTCT |
| | | | | | GG | AT | GG | AT | AGG | T | GG | GGAT |
| 41 | TERT | chr5 | 1295189 | 1295314 | GGCCGCG | CGTCCTG | GGCCGCG | CGTCCTGC | GGCCGCG | GCGTCCTG | GGCCGCG | GCGTCCT |
| | | | | | GAAAGGA | CCCCTTC | GAAAGGA | CCCTTCAC | GAAAGGA | CCCCTTCA | GAAAGGA | GCCCCTT |
| | | | | | AG | ACC | AG | C | AG | CC | AG | CACC |
| 42 | TP53 | chr17 | 7577068 | 7577303 | GCGGAGA | GGCTTCT | GCGGAGA | GGCTTCTC | GCGGAGA | GGCTTCTC | GCGGAGA | GGCTTCT |
| | | | | | TTCTCTTC | CCTCCAC | TTCTCTTC | CTCCACCT | TTCTCTTC | CTCCACCT | TTCTCTTC | CCTCCAC |
| | | | | | CTCTGTG | CTACCT | CTCTGTG | ACCT | CTCTGTG | ACCT | CTCTGTG | CTACCT |
| 43 | TP53 | chr17 | 7576860 | 7577121 | GTGAAAT | CGTGTTT | GTGAAAT | CGTGTTTG | GTGAAAT | GCGTGTTT | GTGAAAT | GCGTGTT |
| | | | | | ATTCTCCA | GTGCCTG | ATTCTCC | TGCCTGTC | ATTCTCC | GTGCCTGT | ATTCTCCA | TGTGCCT |
| | | | | | TCCAGTG | TCCTG | ATCCAGT | CTG | ATCCAGT | CCTG | TCCAGTGG | GTCCTG |
| | | | | | GTTTCT | | GGTTTCT | | GGTTTCT | | TTTCT | |
| 44 | APC | Chr5 | 112175468 | 112175737 | CTTGATA | AAGAACC | CTTGATA | AAGAACC | GCTTGAT | GAAGAACC | GCTTGATA | GAAGAA |
| | | | | | GTTTTGA | TGGACCC | GTTTTGA | TGGACCCT | AGTTTTG | TGGACCCT | GTTTTGAG | CCTGGAC |
| | | | | | GAGTCGT | TCTGAAC | GAGTCGT | CTGAACT | AGAGTCG | CTGAACT | AGTCGTTC | CCTCTGA |
| | | | | | TCGATTG | T | TCGATTG | | TTCGATT | | GATTG | ACT |
| | | | | | | | | | G | | | |
| 45 | APC | Chr5 | 112173802 | 112174055 | AAGAGGA | GTGCATT | AAGAGGA | GTGCATTT | GAAGAGG | GTGCATTT | GAAGAGG | GTGCATT |
| | | | | | AGCTTAG | TCTCTCA | AGCTTAG | CTCTCATC | AAGCTTA | CTCTCATC | AAGCTTAG | TCTCTCA |
| | | | | | ATAGTTCT | TCTGTCA | ATAGTTC | TGTCACAC | GATAGTT | TGTCACAC | ATAGTTCT | TCTGTCA |
| | | | | | CGTTCT | CAC | TCGTTCT | | CTCGTTC | | CGTTCT | CAC |
| | | | | | | | | | T | | | |

TABLE 18
Primers for ploidy detection

| | | Second primer: | Third primer: | Fourth primer: modi- |
|---|---|---|---|---|
| Pri- | First primer: | modified with phos- | with G added | fied with G added to 5′ |
| mer | Ordinary primer | phorylated 5′ end | to 5′ end | end and phosphorylated |
| F | NNNNNNNNNNNNNNN | NNNNNNNNNNNNNNNN | GNNNNNNNNNNNNNN | GNNNNNNNNNNNNNNN |
| | NACACAGGGAGGGGA | ACACAGGGAGGGGAACAT | NNACACAGGGAGGGG | NACACAGGGAGGGGAACAT |
| | ACAT | | AACAT | |
| R | TGCCATGGTGGTTTGCT | TGCCATGGTGGTTTGCT | GTGCCATGGTGGTTTG | GTGCCATGGTGGTTTGCT |
| | | | CT | |

Note:
N represents random primer.

TABLE 19
Primers for fusion detection

| | | | | | | | | Modified with G | |
|---|---|---|---|---|---|---|---|---|---|
| Fu- | | | | Modified with phos- | | With G added to | | added to 5′ end and | |
| sion | Pri- | Ordinary primer | | phorylated 5′ end | | 5′ end | | phosphorylated | |
| type | mer | F | R | F | R | F | R | F | R |
| EML4- | 1 | GAGCAAAA | CATCTGCAT | GAGCAAAA | CATCTGCATG | GAGCAAAA | GCATCTGCA | GAGCAAA | GCATCTGC |
| ALK | | CTACTGTAG | GGCTTGCAG | CTACTGTAG | GCTTGCAGCT | CTACTGTAG | TGGCTTGCA | ACTACTGT | ATGGCTTG |
| | | AGCCCACA | CTCCTGGTG | AGCCCACA | CCTGGTGC | AGCCCACA | GCTCCTGGT | AGAGCCC | CAGCTCCT |
| | | | C | | | | GC | ACA | GGTGC |
| | 2 | GTCCCCAGA | TTGCAGCTC | GTCCCCAGA | TTGCAGCTCC | GTCCCCAGA | GTTGCAGCT | GTCCCCAG | GTTGCAGC |
| | | CAACAAGTA | CTGGTGCTT | CAACAAGT | TGGTGCTTCC | CAACAAGTA | CCTGGTGCT | ACAACAA | TCCTGGTG |
| | | TATAATGT | CCGGCGGTA | ATATAATGT | GCCGGTAC | TATAATGT | TCCGGCGGT | GTATATAA | CTTCCGGC |
| | | | C | | | | AC | TGT | GGTAC |
| | 3 | CATAAAGAT | CTTGCCAGC | CATAAAGA | CTTGCCAGCA | GCATAAAG | GCTTGCCAG | GCATAAA | GCTTGCCA |
| | | GTCATCATC | AAAGCAGT | TGTCATCAT | AAGCAGTAG | ATGTCATCA | CAAAGCAGT | GATGTCAT | GCAAAGC |
| | | AACCAAG | AGTTGGGG | CAACCAAG | TTGGGG | TCAACCAAG | AGTTGGGG | CATCAACC | AGTAGTTG |
| | | | | | | | | AAG | GGG |
| | 4 | AGGAACATT | TACTCAGGG | AGGAACAT | TACTCAGGG | GAGGAACA | GTACTCAGG | GAGGAAC | GTACTCAG |
| | | TAATGATGG | CTCTGCAGC | TTAATGATG | CTCTGCAGCT | TTTAATGAT | GCTCTGCAG | ATTTAATG | GGCTCTGC |
| | | CTTCCAAAT | TCCATCTG | GCTTCCAAA | CCATCTG | GGCTTCCAA | CTCCATCTG | ATGGCTTC | AGCTCCAT |
| | | AG | | TAG | | ATAG | | CAAATAG | CTG |
| | 5 | TTTCTATCC | TCAGCTTGT | TTTCTATCC | TCAGCTTGTA | GTTTCTATC | GTCAGCTTG | GTTTCTAT | GTCAGCTT |
| | | ACACAGAC | ACTCAGGGC | ACACAGAC | CTCAGGGCTC | CACACAGAC | TACTCAGGG | CCACACA | GTACTCAG |
| | | GGGAATG | TCTGCAGC | GGGAATG | TGCAGC | GGGAATG | CTCTGCAGC | GACGGGA | GGCTCTGC |
| | | | | | | | | ATG | AGC |
| | 6 | ATCATGTGG | GCTCCTGGT | ATCATGTGG | GCTCCTGGTG | GATCATGTG | GCTCCTGGT | GATCATGT | GCTCCTGG |
| | | CCTCAGTGA | GCTTCCGGC | CCTCAGTGA | CTTCCGGCGG | GCCTCAGTG | GCTTCCGGC | GGCCTCA | TGCTTCCG |
| | | AAAAATC | GGTA | AAAAATC | TA | AAAAAATC | GGTA | GTGAAAA | GCGGTA |
| | | | | | | | | AATC | |
| | 7 | CTGTGATGC | TCCTGGTGC | CTGTGATGC | TCCTGGTGCT | GCTGTGATG | GTCCTGGTG | GCTGTGAT | GTCCTGGT |
| | | GCTACTCAA | TTCCGGCGG | GCTACTCAA | TCCGGCGGT | CGCTACTCA | CTTCCGGCG | GCGCTACT | GCTTCCGG |
| | | TAG | T | TAG | | ATAG | GT | CAATAG | CGGT |
| | 8 | CCTCAGTGA | GGCTTGCAG | CCTCAGTGA | GGCTTGCAG | GCCTCAGTG | GGCTTGCAG | GCCTCAGT | GGCTTGCA |
| | | AAAAATCA | CTCCTGGTG | AAAAATCA | CTCCTGGTG | AAAAAATC | CTCCTGGTG | GAAAAAA | GCTCCTGG |
| | | GTCTCAAG | | GTCTCAAG | | AGTCTCAAG | | TCAGTCTC | TG |
| | | | | | | | | AAG | |
| | 9 | TTAATGATG | GGCTTGCAG | TTAATGATG | GGCTTGCAG | GTTAATGAT | GGCTTGCAG | GTTAATG | GGCTTGCA |
| | | GCTTCCAAA | CTCCTGGTG | GCTTCCAAA | CTCCTGGTGC | GGCTTCCAA | CTCCTGGTG | ATGGCTTC | GCTCCTGG |
| | | TAGAAGTA | CTTCCG | TAGAAGTA | TTCCG | ATAGAAGTA | CTTCCG | CAAATAG | TGCTTCCG |
| | | | | | | | | AAGTA | |
| | 10 | CCCACACCT | ATCTGCATG | CCCACACCT | ATCTGCATGG | GCCCACACC | GATCTGCAT | GCCCACA | GATCTGCA |
| | | GGGAAAGG | GCTTGCAGC | GGGAAAGG | CTTGCAGCTC | TGGGAAAG | GGCTTGCAG | CCTGGGA | TGGCTTGC |
| | | ACCTA | TC | ACCTA | | GACCTA | CTC | AAGGACC | AGCTC |
| | | | | | | | | TA | |
| | 11 | CTGAATCCT | TGCATGGCT | CTGAATCCT | TGCATGGCTT | GCTGAATCC | GTGCATGGC | GCTGAAT | GTGCATGG |
| | | GAAAGAGA | TGCAGCTCC | GAAAGAGA | GCAGCTCCTG | TGAAAGAG | TTGCAGCTC | CCTGAAA | CTTGCAGC |
| | | AATAGAG | TGGTG | AATAGAG | GTG | AAATAGAG | CTGGTG | GAGAAAT | TCCTGGTG |
| | | | | | | | | AGAG | |
| NPM1- | 12 | CCAGTGCAT | GCAGCTCCT | CCAGTGCAT | GCAGCTCCTG | GCCAGTGCA | GCAGCTCCT | GCCAGTG | GCAGCTCC |
| ALK | | ATTAGTGGA | GGTGCTTCC | ATTAGTGGA | GTGCTTCCGG | TATTAGTGG | GGTGCTTCC | CATATTAG | TGGTGCTT |
| | | CAGCA | GG | CAGCA | | ACAGCA | GG | TGGACAG | CCGG |
| | | | | | | | | CA | |
| CLTC- | 13 | AAGGAGTA | GCATGGCTT | AAGGAGTA | GCATGGCTTG | GAAGGAGT | GCATGGCTT | GAAGGAG | GCATGGCT |
| ALK | | CTTGACAAA | GCAGCTCCT | CTTGACAAA | CAGCTCCTGG | ACTTGACAA | GCAGCTCCT | TACTTGAC | TGCAGCTC |
| | | G | GGTGCTT | G | TGCTT | AG | GGTGCTT | AAAG | CTGGTGCT |
| | 14 | AAGAAGAA | TCTGCAGCT | AAGAAGAA | TCTGCAGCTC | GAAGAAGA | GTCTGCAGC | GAAGAAG | GTCTGCAG |
| | | CAAGCTACA | CCATCTGCA | CAAGCTAC | CATCTGCATG | ACAAGCTAC | TCCATCTGC | AACAAGC | CTCCATCT |
| | | GAGACACA | TGGC | AGAGACAC | GC | AGAGACAC | ATCGC | TACAGAG | GCATGGC |
| | | ACCCAT | | AACCCAT | | AACCCAT | | ACACAAC | |
| | | | | | | | | CCAT | |
| CCDC | 15 | CGCAAAGC | TCTTCACGG | CGCAAAGC | TCTTCACGGC | GCGCAAAG | GTCTTCACG | GCGCAAA | GTCTTCAC |
| 6-RET | | AAAGCCAG | CCACCGTGG | AAAGCCAG | CACCGTGGT | CAAAGCCA | GCCACCGTG | GCAAAGC | GGCCACC |
| | | CGTGACCAT | TGTACCCTG | CGTGACCAT | GTACCCTGC | GCGTGACCA | GTGTACCCT | CAGCGTG | GTGGTGTA |
| | | C | C | C | | TC | GC | ACCATC | CCCTGC |
| | 16 | AAGAATTCC | TGGTGTACC | AAGAATTCC | TGGTGTACCC | GAAGAATTC | GTGGTGTAC | GAAGAAT | GTGGTGTA |
| | | TCACTAATG | CTGCTCTGC | TCACTAATG | TGCTCTGCCT | CTCACTAAT | CCTGCTCTG | TCCTCACT | CCCTGCTC |
| | | AGCTCTCCA | CTTTCAGAT | AGCTCTCCA | TTCAGAT | GAGCTCTCC | CCTTTCAGA | AATGAGC | TGCCTTTC |
| | | G | | G | | AG | T | TCTCCAG | AGAT |
| | 17 | GCAGCACAT | TCCACGGAG | GCAGCACA | TCCACGGAG | GCAGCACAT | GTCCACGGA | GCAGCAC | GTCCACGG |
| | | GGGAACATC | ACCTGGTTC | TGGGAACA | ACCTGGTTCT | GGGAACATC | GACCTGGTT | ATGGGAA | AGACCTGG |
| | | CCATGGTAT | TCCATGGAG | TCCCATGGT | CCATGGAGT | CCATGGTAT | CTCCATGGA | CATCCCAT | TTCTCCAT |
| | | C | TC | ATC | C | C | GTC | GGTATC | GGAGTC |
| NCOA | 18 | GCTTACCCA | TCACGGCCA | GCTTACCCA | TCACGGCCA | GCTTACCCA | GTCACGGCC | GCTTACCC | GTCACGGC |
| 4-RET | | AAAGCAGA | CCGTGGTGT | AAAGCAGA | CCGTGGTGTA | AAAGCAGA | ACCGTGGTG | AAAAGCA | CACCGTGG |
| | | CCTTGGAGA | ACCCTGCTC | CCTTGGAGA | CCCTGCTCTG | CCTTGGAGA | TACCCTGCT | GACCTTGG | TGTACCCT |
| | | AC | TG | AC | | AC | CTG | AGAAC | GCTCTG |
| | 19 | GTGGATTCT | TGAGGGCA | GTGGATTCT | TGAGGGCAA | GTGGATTCT | GTGAGGGCA | GTGGATTC | GTGAGGG |
| | | AGTAGTGTG | AATGTTGAT | AGTAGTGTG | ATGTTGATGT | AGTAGTGTG | AATGTTGAT | TAGTAGTG | CAAATGTT |
| | | GATTCTAGT | GTCTTGGGT | GATTCTAGT | CTTGGGTCT | GATTCTAGT | GTCTTGGGT | TGGATTCT | GATGTCTT |
| | | AGTTTATAT | CT | AGTTTATAT | | AGTTTATAT | CT | AGTAGTTT | GGGTCT |
| | | ACC | | ACC | | ACC | | ATATACC | |
| KIF5B- | 20 | GAATTGCTG | GCCACCGTG | GAATTGCTG | GCCACCGTG | GAATTGCTG | GCCACCGTG | GAATTGCT | GCCACCCT |
| RET | | TGGGAAATA | GTGTACCCT | TGGGAAAT | GTGTACCCTG | TGGGAAATA | GTGTACCCT | GTGGGAA | GGTGTACC |
| | | ATGATGT | GCTCTGC | AATGATGT | CTCTGC | ATGATGT | GCTCTGC | ATAATGAT | CTGCTCTG |
| | | | | | | | | GT | C |
| | 21 | GAGTTAGCA | TCTTCACGG | GAGTTAGC | TCTTCACGGC | GAGTTAGCA | GTCTTCACG | GAGTTAG | GTCTTCAC |
| | | GCATGTCAG | CCACCGTGG | AGCATGTCA | CACCGTGGT | GCATGTCAG | GCCACCGTG | CAGCATGT | GGCCACC |
| | | CTTCGTATC | TCTACCCT | GCTTCGTAT | GTACCCT | CTTCGTATC | GTGTACCCT | CAGCTTCG | GTGGTGTA |
| | | TCT | | CTCT | | TCT | | TATCTCT | CCCT |
| | 22 | TTCAGGACC | TGGTGTACC | TTCAGGACC | TGGTGTACCC | GTTCAGGAC | GTGGTGTAC | GTTCAGG | GTGGTGTA |
| | | TGGCTACAA | CTGCTCTGC | TGGCTACAA | TGCTCTGCCT | CTGGCTACA | CCTGCTCTG | ACCTGGCT | CCCTGCTC |
| | | GAGTTAA | CTTTCAGAT | GAGTTAA | TTCAGAT | AGAGTTAA | CCTTTCAGA | ACAAGAG | TGCCTTTC |
| | | | | | | | T | TTAA | AGAT |
| CD74- | 23 | CCCACCCAC | CTCAAGGAT | CCCACCCAC | CTCAAGGAT | GCCCACCCA | GCTCAAGGA | GCCCACC | GCTCAAG |
| ROS1 | | TGACGCTCC | ATAGTATGT | TGACGCTCC | ATAGTATGTA | CTGACGCTC | TATAGTATG | CACTGAC | GATATAGT |
| | | ACCGAA | AATTCTACA | ACCGAA | ATTCTACAT | CACCGAA | TAATTCTAC | GCTCCACC | ATGTAATT |
| | | | T | | | | AT | GAA | CTACAT |
| | 24 | AGCAGGCA | TGTAACAAC | AGCAGGCA | TCTAACAAC | GAGCAGGC | GTGTAACAA | GAGCAGG | GTGTAACA |
| | | CTCCTTGGA | CAGAAATAT | CTCCTTGGA | CAGAAATAT | ACTCCTTGG | CCAGAAATA | CACTCCTT | ACCAGAA |
| | | GCAAAAGC | TCCAACTA | GCAAAAGC | TCCAACTA | AGCAAAAG | TTCCAACTA | GGAGCAA | ATATTCCA |
| | | CC | | CC | | CCC | | AAGCCC | ACTA |
| SLC34 | 25 | GTAGCGCCT | AAGGATAT | GTAGCGCCT | AAGGATATA | GTAGCGCCT | GAAGGATAT | GTAGCGC | GAAGGAT |
| A2- | | TCCAGCTGG | AGTATGTAA | TCCAGCTGG | GTATGTAATT | TCCAGCTGG | AGTATGTAA | CTTCCAGC | ATAGTATG |
| ROS1 | 26 | TTGGA | TTCTACATC | TTGGA | CTACATCCA | TTGGA | TTCTACATCC | TCGTTGGA | TAATTCTA |
| | | | CA | | | | A | | CATCCA |
| | | ACAGGCGTG | GGATATAGT | ACAGGCGT | GGATATAGT | GACAGGCG | GGATATAGT | GACAGGC | GGATATAG |
| | | AGCCACCAC | ATGTAATTC | GAGCCACC | ATGTAATTCT | TGAGCCACC | ATGTAATTC | GTGAGCC | TATGTAAT |
| | | CAGGCCTGA | TACA | ACCAGGCCT | ACA | ACCAGGCCT | TACA | ACCACCA | TCTACA |
| | | | | GA | | GA | | GGCCTGA | |
| TPM3- | 27 | CTGGAAAA | CCAGGTCAT | CTGGAAAA | CCAGGTCATC | GCTGGAAA | GCCAGGTCA | GCTGGAA | GCCAGGTC |
| NTRK1 | | GACAATTGA | CAATTGTCT | GACAATTG | AATTGTCTTT | AGACAATTG | TCAATTGTCT | AAGACAA | ATCAATTG |
| | | TGACCTGG | TTTCCAG | ATGACCTGG | TCCAG | ATGACCTGG | TTTCCAG | TTGATGAC | TCTTTTCC |
| | | | | | | | | CTGG | AG |
| PAX8- | 28 | GCCACACCC | CCAGAATG | GCCACACCC | CCAGAATGG | GCCACACCC | GCCAGAATG | GCCACAC | GCCAGAA |
| PPARG | | CCTACTCCT | GCATCTCTG | CCTACTCCT | CATCTCTGTG | CCTACTCCT | GCATCTCTG | CCCCTACT | TGGCATCT |
| | | CCTACAGC | TG | CCTACAGC | | CCTACAGC | TG | CCTCCTAC | CTGTG |
| | | | | | | | | AGC | |
| | 29 | CCTCCGTGT | AGAATGGC | CCTCCGTGT | AGAATGGCA | GCCTCCGTG | GAGAATGGC | GCCTCCGT | GAGAATG |
| | | ACGGGCAGT | ATCTCTGTG | ACGGGCAG | TCTCTGTGTC | TACGGGCAG | ATCTCTGTGT | GTACGGG | GCATCTCT |
| | | TCACGGGCC | TCAACCA | TTCACGGGC | AACCA | TTCACGGGC | CAACCA | CAGTTCAC | GTGTCAAC |
| | | A | | CA | | CA | | GCGCCA | CA |
| | 30 | GCCTCCCCC | TGGTGGGCC | GCCTCCCCC | TGGTGGGCC | GCCTCCCCC | GTGGTGGGC | GCCTCCCC | GTGGTGG |
| | | AGCCACACC | AGAATGGC | AGCCACAC | AGAATGGCA | AGCCACACC | CAGAATGGC | CAGCCAC | GCCAGAAT |
| | | AAAGGCGA | ATCTC | CAAAGGCG | TCTC | AAAGGCGA | ATCTC | ACCAAAG | GGCATCTC |
| | | G | | AG | | G | | GCGAG | |
| | 31 | CCTCCTCTG | CCAGAATG | CCTCCTCTG | CCAGAATGG | GCCTCCTCT | GCCAGAATG | GCCTCCTC | GCCAGAA |
| | | CCATCGCAG | GCATCTCTG | CCATCGCAG | CATCTCTGTG | GCCATCGCA | GCATCTCTG | TGCCATCG | TGGCATCT |
| | | GCATGGTG | TGTCA | GCATGGTG | TCA | GGCATGGTG | TGTCA | CAGGCAT | CTGTGTCA |
| | | | | | | | | GGTG | |
| ETV6- | 32 | GTCTCTGTC | CACGATGTC | GTCTCTGTC | CACGATGTCT | GTCTCTGTC | GCACGATGT | GTCTCTGT | GCACGAT |
| NTRK3 | | TCCCCGCCT | TCTCCTCTT | TCCCCGCCT | CTCCTCTTAA | TCCCCGCCT | CTCTCCTCTT | CTCCCCGC | GTCTCTCC |
| | | GA | AATG | GA | TG | GA | AATG | CTGA | TCTTAATG |
| | 33 | CCGGAGGTC | TTGATGTGG | CCGGAGGT | TTGATGTGGT | GCCGGAGG | GTTGATGTG | GCCGGAG | GTTGATGT |
| | | ATACTGCAT | TGCAGTGGG | CATACTGCA | GCAGTGGG | TCATACTGC | GTGCAGTGG | GTCATACT | GGTGCAGT |
| | | CAGA | | TCAGA | | ATCAGA | G | GCATCAG | GGG |
| | | | | | | | | A | |

TABLE 20
Primers for pathogen detection

| Pathogenic | | | With phosphor- | | With G added to | | With G added to 5′ end | |
|---|---|---|---|---|---|---|---|---|
| micro- | Ordinary primer | | ylated 5′ end | | 5′ end | | and phosphorylated | |
| organism | F | R | F | R | F | R | F | R |
| Influenza A | AATGGCT | GACAAAGC | AATGGCT | GACAAAG | GAATGGCTA | GACAAAG | GAATGGC | GACAAAG |
| virus | AAAGACA | GTCTACGC | AAAGACA | CGTCTACG | AAGACAAG | CGTCTAC | TAAAGAC | CGTCTAC |
| | AGACCA | TGCAG | AGACCA | CTGCAG | ACCA | GCTGCAG | AAGACCA | GCTGCAG |
| HINI | ATTATGA | ACATGCTG | ATTATGA | ACATGCTG | GATTATGAG | GACATGC | GATTATG | GACATGC |
| | GGAGCTA | CCGTTACA | GGAGCTA | CCGTTACA | GAGCTAAGA | TGCCGTT | AGGAGCT | TGCCGTT |
| | AGAGAG | CC | AGAGAG | CC | GAG | ACACC | AAGAGAG | ACACC |
| H3N2 | GAGAAAA | TAACAGTT | GAGAAAA | TAACAGTT | GAGAAAACT | GTAACAG | GAGAAAA | GTAACAG |
| | CTGCACA | GCTGTAGG | CTGCACA | GCTGTAG | GCACACTAA | TTGCTGT | CTGCACA | TTGCTGT |
| | CTAATAG | CTTT | CTAATAG | GCTTT | TAGATG | AGGCTTT | CTAATAG | AGGCTTT |
| | ATG | | ATG | | | | ATG | |
| H7N9 | AAAATGT | CTATAGCA | AAAATGT | CTATAGCA | GAAAATGTC | GCTATAG | GAAAATG | GCTATAG |
| | CCGAGAT | CCAAATAG | CCGAGAT | CCAAATA | CGAGATATG | CACCAAA | TCCGAGA | CACCAAA |
| | ATGT | GCCTC | ATGT | GGCCTC | T | TAGGCCT | TATGT | TAGGCCT |
| | | | | | | C | | C |
| influenza B | TAAGATG | GTGTCTTG | TAAGATG | GTGTCTTG | GTAAGATGT | GTGTCTT | GTAAGAT | GTGTCTT |
| virus | TGGCGAA | AGAAAATA | TGGCGAA | AGAAAAT | GGCGAATGC | GAGAAA | GTGGCGA | GAGAAAA |
| | TGCA | CCA | TGCA | ACCA | A | ATACCA | ATGCA | TACCA |
| Influenza C | CTTCTGCT | CTAATCCC | CTTCTGCT | CTAATCCC | GCTTCTGCT | GCTAATC | GCTTCTG | GCTAATC |
| virus | TGCAATC | AAAGAGGC | TGCAATCT | AAAGAGG | TGCAATCTA | CCAAAGA | CTTGCAA | CCAAAGA |
| | TAAA | TAATG | AAA | CTAATG | AA | GGCTAAT | TCTAAA | GGCTAAT |
| | | | | | | G | | G |
| Human para- | AGTTATG | GATGGTCA | AGTTATG | GATGGTC | GAGTTATGC | GATGGTC | GAGTTAT | GATGGTC |
| influenza | CTCCTTG | AAAGTTAT | CTCCTTGC | AAAAGTT | TCCTTGCCC | AAAAGTT | GCTCCTT | AAAAGTT |
| virus 1 | CCCAC | ATCTTC | CCAC | ATATCTTC | AC | ATATCTT | GCCCAC | ATATCTT |
| | | | | | | C | | C |
| Human Para- | TCAAAGT | TCCCTTTA | TCAAAGT | TCCCTTTA | GTCAAAGTC | GTCCCTT | GTCAAAG | GTCCCTT |
| influenza | CTTCGAG | AGAGCTCA | CTTCGAGT | AGAGCTC | TTCGAGTGG | TAAGAGC | TCTTCGA | TAAGAGC |
| virus 2 | TGGTGTA | ATGAT | GGTGTA | AATGAT | TGTA | TCAATGA | GTGGTGT | TCAATGA |
| | | | | | | T | A | T |
| Human Para- | CAAAATC | ATCTTCAT | CAAAATC | ATCTTCAT | GCAAAATCA | GATCTTC | GCAAAAT | GATCTTC |
| influenza | ATTAATT | ATCTGATT | ATTAATTC | ATCTGATT | TTAATTCGG | ATATCTG | CATTAAT | ATATCTG |
| virus 3 | CGGGTTG | TTATCAC | GGGTTGG | TTATCAC | GTTGG | ATTTTAT | TCGGGTT | ATTTTAT |
| | G | | | | | CAC | GG | CAC |
| Human Para- | TTAACGG | GGGACATC | TTAACGG | GGGACAT | GTTAACGGA | GGGACAT | GTTAACG | GGGACAT |
| influenza | AAGAACC | TTCCACAT | AAGAACC | CTTCCACA | AGAACCACA | CTTCCAC | GAAGAAC | CTTCCAC |
| virus 4 | ACAAT | GTGCATTT | ACAAT | TGTGCATT | AT | ATGTGCA | CACAAT | ATGTGCA |
| | | TTCG | | TTTCG | | TTTTTCG | | TTTTTCG |
| Respiratory | CAAATAT | GCACCCAT | CAAATAT | GCACCCAT | GCAAATATG | GCACCCA | GCAAATA | GCACCCA |
| syncytial | GGAAACA | ATTGTAAG | GGAAACA | ATTGTAAG | GAAACATAC | TATTGTA | TGGAAAC | TATTGTA |
| virus | TACGTGA | TGATGC | TACGTGA | TGATGC | GTGAAC | AGTGATG | ATACGTG | AGTGATG |
| | AC | | AC | | | C | AAC | C |
| Human | GGGATCA | TATACACA | GGGATCA | TATACACA | GGGATCAAT | GTATACA | GGGATCA | GTATACA |
| coronavirus | ATTCCTTT | TTCACCGT | ATTCCTTT | TTCACCGT | TCCTTTAC | CATTCAC | ATTCCTTT | CATTCAC |
| 229E | AC | TATA | AC | TATA | | CGTTATA | AC | CGTTATA |
| Human | TGTCGCA | TATTACCA | TGTCGCA | TATTACCA | GTGTCGCAA | GTATTAC | GTGTCGC | GTATTAC |
| coronavirus | AGTTTGG | TAAGTAGT | AGTTTGG | TAAGTAGT | GTTTGGCA | CATAAGT | AAGTTTG | CATAAGT |
| HKUI | CA | AAAAC | CA | AAAAC | | AGTAAAA | GCA | AGTAAAA |
| | | | | | | C | | C |
| human | ATTGCAC | TATTTCAA | ATTGCAC | TATTTCAA | GATTGCACC | GTATTTC | GATTGCA | GTATTTC |
| coronavirus | CATAGCT | GTCTAGCC | CATAGCT | GTCTAGCC | ATAGCTCAA | AAGTCTA | CCATAGC | AAGTCTA |
| oc43 | CAACTC | GGTGAT | CAACTC | GGTGAT | CTC | GCCGGTG | TCAACTC | GCCGGTG |
| | | | | | | AT | | AT |
| Human | GTTTTGTC | TTAGTTTC | GTTTTGTC | TTAGTTTC | GTTTTGTCA | GTTAGTT | GTTTTGTC | GTTAGTT |
| coronavirus | AATTTTA | AGGATTAA | AATTTTAA | AGGATTA | ATTTTAATG | TCAGGAT | AATTTTA | TCAGGAT |
| NL63 | ATGTG | AAGC | TGTG | AAAGC | TG | TAAAAGC | ATGTG | TAAAAGC |
| MERS | CTTATGTT | AGCTCTAA | CTTATGTT | AGCTCTAA | GCTTATGTT | GAGCTCT | GCTTATG | GAGCTCT |
| coronavirus | ATGCACA | CTTCTTTGT | ATGCACA | CTTCTTTG | ATGCACAAC | AACTTCT | TTATGCA | AACTTCT |
| | ACTATG | AGAA | ACTATG | TAGAA | TATG | TTGTAGA | CAACTAT | TTGTAGA |
| | | | | | | A | G | A |
| SARS | GGCTTTG | ACGACAAT | GGCTTTGT | ACGACAA | GGCTTTGTT | GACGACA | GGCTTTG | GACGACA |
| coronavirus | TTGGAAG | TGTATCTG | TGGAAGT | TTGTATCT | GGAAGTGCA | ATTGTAT | TTGGAAG | ATTGTAT |
| | TGCAA | TGA | GCAA | GTGA | A | CTGTGA | TGCAA | CTGTGA |
| covid-19 | GTCTGCG | TGCCTGTG | GTCTGCG | TGCCTGTG | GTCTGCGGT | GTGCCTG | GTCTGCG | GTGCCTG |
| | GTATGTG | CCGCACGG | GTATGTG | CCGCACG | ATGTGGAAA | TGCCGCA | GTATGTG | TGCCGCA |
| | GAAAGG | TGTAAGAC | GAAAGG | GTGTAAG | GG | CGGTGTA | GAAAGG | CGGTGTA |
| | | | | AC | | AGAC | | AGAC |

TABLE 21
TA cloning primers

| Primer type | F | R |
|---|---|---|
| ordinary primer | ATGCCCTTCCAGCCAACTCATATACT | AGGCACGTACCGTTTGCACCATATT |
| Primers with | ATGCCCTTCCAGCCAACTCATATACT | AGGCACGTACCGTTTGCACCATATT |
| phosphorylated 5′ end | | |
| Primers with G added | GATGCCCTTCCAGCCAACTCATATACT | GAGGCACGTACCGTTTGCACCATATT |
| to 5′ end | | |
| Primers with G added | GATGCCCTTCCAGCCAACTCATATACT | GAGGCACGTACCGTTTGCACCATATT |
| to 5′ end and | | |
| phosphorylated | | |

Each of the technical features of the above-mentioned embodiments may be combined arbitrarily. To simplify the description, not all the possible combinations of each of the technical features in the above embodiments are described. However, all of the combinations of these technical features should be considered as within the scope of this disclosure, as long as such combinations do not contradict with each other.

The aforementioned embodiments only illustrate several embodiments of the present disclosure, which facilitate a specific and detailed understanding of the technical solutions of the present disclosure, but they cannot be understood to limit the protection scope of the present disclosure. It should be noted that a plurality of variations and modifications may be made by those skilled in the art without departing from the conception of the present disclosure, which are all within the scope of protection of the present disclosure. Accordingly, the scope of protection of the present disclosure shall be based on the appended claims, and the description may be used to interpret the content of the claims.

1. An amplification primer for sequencing library construction comprising a primer sequence fragment complementary to a target fragment and a base G ligated to the 5′ end of the primer sequence fragment, wherein the amplification primer and the target fragment are not complementary at the base G.

2. The amplification primer according to claim 1, wherein the base G is directly ligated to the 5′ end of the primer sequence fragment.

3. The amplification primer according to claim 1, wherein the base G is a base modified by phosphorylation.

4. (canceled)

5. A method for constructing a sequencing library, comprising:
providing a primer sequence fragment used for performing amplification and library construction on a deoxyribonucleic acid, the primer sequence fragment being complementary to a target fragment,
performing a treatment on the primer sequence fragment to obtain an amplification primer with a base G at the 5′ end, wherein the treatment includes:
ligating a base G to the 5′ end of the primer sequence fragment if the base at the 5′ end of the primer sequence fragment is not G, or
ligating or not ligating a base G to the 5′ end of the primer sequence fragment if the base at the 5′ end of the primer sequence fragment is G;
performing cyclic amplification on the deoxyribonucleic acid using the amplification primer, to obtain an amplification fragment with a base C at the 3′ end;
adding a base A to the 3′ end of the amplification fragment; and
ligating an adaptor containing a T sticky end to the amplification fragment with base A added to the 3′ end, wherein the adaptor contains a sequence required by a sequencing platform.

6. The method for constructing a sequencing library according to claim 5, further comprising: phosphorylating the base G at the 5′ end of the amplification primer before performing amplification on the deoxyribonucleic acid.

7. The method for constructing a sequencing library according to claim 5, wherein the deoxyribonucleic acid is selected from the group consisting of a sample DNA, a sample plasmid, and a deoxyribonucleic acid obtained by reverse transcription of a sample RNA.

8. A sequencing library constructed by the method for constructing a sequencing library according to claim 5.

9. A kit for constructing a sequencing library, comprising the amplification primer according to claim 1.

10. The kit according to claim 9, further comprising at least one of a reagent for adding base A, a reagent for adding adaptors, a reagent for PCR amplification and a reagent for purification.

11. A sequencing method, comprising: constructing a sequencing library for a sample using the method for constructing a sequencing library according to claim 5, and performing sequencing.

12. The sequencing method according to claim 11, wherein the sequencing method is used to perform on the sample any one of mutation site detection, chromosome ploidy detection, gene fusion detection, and pathogenic microorganism detection.

13. The sequencing method according to claim 12, wherein the mutation site detection comprises at least one of intestinal cancer mutation sites, cervical cancer mutation sites, and urothelial cancer mutation sites.

14. The sequencing method according to claim 12, wherein the chromosome ploidy comprises at least one of the chromosome ploidy of intestinal cancer, the chromosome ploidy of cervical cancer, and the chromosome ploidy of urothelial cancer.

15. The sequencing method according to claim 12, wherein the gene fusion comprises at least one of EML4-ALK gene fusion, CD74-ROSI gene fusion, CCDC6-RET gene fusion, NCOA4-RET gene fusion, and TPM3-NTRK1 gene fusion.

16-17. (canceled)

18. A mutation site detection kit, comprising the amplification primer according to claim 1.

19. A chromosome ploidy detection kit, comprising the amplification primer according to claim 1.

20. A gene fusion detection kit, comprising the amplification primer according to claim 1.

21. A pathogenic microorganism detection kit, comprising the amplification primer according to claim 1.

22. A TA cloning method, comprising the steps: performing PCR amplification using the amplification primer according to claim 1 to add a base A to the end of an amplification product.

23. A method for diagnosing a disease in a subject, comprising collecting a biological sample from the subject, and performing sequencing on a target relating to the disease in the biological sample using the sequencing method of claim 11, and obtaining a diagnosis result of the disease based on the sequencing result of the target.
